SEQUENCE LISTING (32 sequences)

SEQ ID NO: 1 - DNA, 21 bp, Streptomyces avidinii
gatccgacccctccaaggact

SEQ ID NO: 2 - DNA, 31 bp, Streptomyces avidinii
gcgcagatctcgagctgctgaacggcgtcga

SEQ ID NO: 3 - DNA, 31 bp, Artificial Sequence
  Artificial sequence for creation of linker
catgggcagcagccatcatcatcatcatcac

SEQ ID NO: 4 - DNA, 31 bp, Artificial Sequence
  Artificial sequence for creation of linker
cggcgtgatgatgatgatgatggctgctgcc

SEQ ID NO: 5 - DNA, 19 bp, Artificial Sequence
  Artificical sequence to serve as T7 primer
taatacgactcactatagg

SEQ ID NO: 6 - DNA, 39 bp, Artificial Sequence
  Artificial sequence for glycine-serine double stranded oligo
tcgacggatcgggcggtggctccggtggcggcagcggcc

SEQ ID NO: 7 - DNA, 39 bp, Artificial Sequence
  Artificial sequence as lower strand for double stranded oligo
tcgaggccgctgccgccaccggagccaccgcccgatccg

SEQ ID NO: 8 - DNA, 31 bp, E. coli
gcgcgtcgacgaagaaaaattcggtgtttcc

SEQ ID NO: 9 - DNA, 32 bp, E. coli
gcgcctcgagcttttcttcagcagcttcaacc

SEQ ID NO: 10 - DNA, 42 bp, Artificial Sequence
  Oligo for encoding TEV protease site
tcgaaggcaccacagagaacctgtactttcagagcggcaccc

SEQ ID NO: 11 - DNA, 42 bp, Artificial Sequence
  Oligo for creating TEV protease cleavage site
tcgagggtgccgctctgaaagtacaggttctctgtggtgcct

SEQ ID NO: 12 - DNA, 33 bp, Artificial Sequence
  Oligo for creating Precission cleavage site
tcgatgtgctgtttcaaggtccaggatccggtc

SEQ ID NO: 13 - DNA, 33 bp, Artificial Sequence
  Oligo for creating Prescission cleavage site
tcgagaccggatcctggaccttgaaacagcaca

SEQ ID NO: 14 - DNA, 50 bp, E. coli
gcgcgtcgacggtggcggtggctcggtgatttcttatgacaactacgtca

SEQ ID NO: 15 - DNA, 32 bp, E. coli
gcgcaagcttagtgcgcctgatcccagttttc

SEQ ID NO: 16 - DNA, 56 bp, Artificial Sequence
  Oligo for creating flag tag
ctagtgattataaggatgacgatgacaaagattacaaagatgatgacgataagtag

SEQ ID NO: 17 - DNA, 56 bp, Artificial Sequence
  Oligo for creating flag tag
aattctacttatcgtcatcatctttgtaatctttgtcatcgtcatccttataatca

SEQ ID NO: 18 - DNA, 20 bp, Artificial Sequence
  Primer to remove stop codon from Klenow
cgtgcactcgagttgctaaa

SEQ ID NO: 19 - DNA, 31 bp, Artificial Sequence
  Primer to remove stop codon of Klenow
gcgcactagtatgcgcctgatcccagttttc

SEQ ID NO: 20 - DNA, 23 bp, Artificial Sequence
  Primer for sequencing plasmids
ctacttgtcatcgtcatccttat

SEQ ID NO: 21 - DNA, 20 bp, Artificial Sequence
  Primer for sequencing flag tags
cggcagaagtgtttggtttg

SEQ ID NO: 22 - DNA, 29 bp, Geobacillus thermodenitrificans
gcgcctcgagcatggcggacaagcagtat

SEQ ID NO: 23 - DNA, 30 bp, Geobacillus thermodenitrificans
gcgcaagcttctatggcctcacgacacatt

SEQ ID NO: 24 - DNA, 85 bp, Artificial Sequence
  Oligo for sequencing cleavage products
tttttttgctcgacggtgaangangtaggnaccagcagnaacncgagcacgncnncmgacmcaaatcngganattgcagcctcgt

SEQ ID NO: 25 - DNA, 20 bp, Artificial Sequence
  Primer for sequencing cleavage products
acgaggctgcaatatccaga

SEQ ID NO: 26 - DNA, 20 bp, Artificial Sequence
  Primer for sequencing
tgctcgacggtgaatgatgt

SEQ ID NO: 27 - DNA, 20 bp, Artificial Sequence
  Reverse primer for sequencing
acgaggctgcaatatccaga

SEQ ID NO: 28 - protein, 5 aa, Artificial Sequence
  Enterokinase cleavage site
DDDDK

SEQ ID NO: 29 - protein, 4 aa, Artificial Sequence
  Factor Xa protease cleavage site
IXGR

SEQ ID NO: 30 - protein, 6 aa, Artificial Sequence
  Thrombin consensus cleavage site
LVPRGS

SEQ ID NO: 31 - protein, 7 aa, Artificial Sequence
  TEV protease consensus cleavage site
ENLYFQG

SEQ ID NO: 32 - protein, 8 aa, Artificial Sequence
  Prescission protease cleavage site
LEVLFQGP

(19) United States
(12) Patent Application Publication
(10) Pub. No.: US 2009/0298088 A1
(43) Pub. Date: Dec. 3, 2009

(54) CLEAVABLE CATALYTIC BINDING AND DETECTION SYSTEM

(76) Inventors: Alexander S. BELYAEV, San Diego, CA (US); Craig R. Monell, La Jollas, CA (US); Katherine A. Felts, San Diego, CA (US)

Correspondence Address: AGILENT TECHOLOGIES INC, P.O BOX 7599, BLDG E , LEGAL, LOVELAND, CO, 80537-0599, US

(21) Appl. No.: 12/129,800

(22) Filed: May 30, 2008

(51) Int. Cl. G01N 33/53

(52) U.S. Cl. 435/7.1

(57) ABSTRACT

The present invention provides a detection reagent for detection of the presence of a substance of interest in a sample. The detection reagent comprises a binding portion, a linking portion, and a catalytic portion. The linking portion comprises a cleavage site for cleavage of the binding portion from the catalytic portion. According to the method, the detection reagent is caused to bind to the substance of interest. The bound reagent is then cleaved by breaking of a bond in the linking portion. Upon cleavage, the catalytic portion is removed from the binding reaction mixture and caused to catalyze a reaction that produces a detectable product.

### BACKGROUND OF THE INVENTION

1. Field of the Invention

The present invention relates to the field of biotechnology. More specifically, it relates to methods for detecting substances using binding moieties that specifically bind to the substances, where the binding moieties are linked to catalytic moieties that provide a means of detection to form a complex that can be cleaved to release the binding moiety from the detection or catalytic moiety. The combination of a specific binding reaction and a supplemental catalytic detection step allows for sensitive and quantitative detection of substances of interest.

2. Description of Related Art

Traditional methods for protein detection include gel electrophoresis, mass spectrometry, and antibody binding, such as enzyme-linked immunosorbent assays (ELISAs). In a standard ELISA assay, an unknown amount of antigen is affixed to a surface, a specific labeled antibody is washed over the surface so that it can bind to the antigen, and the labeled antibody, bound to the surface, is detected. One common label, horseradish peroxidase, reacts with appropriate substrates (such as ABTS or 3,3′,5,5′-Tetramethylbenzidine), resulting in changes in color, which are measured as a signal. Problems with this method include high background levels as well as low sensitivity. For example, direct binding of horseradish peroxidase to an ELISA plate results in unspecific background signal. To reduce unspecific binding, blocking solutions containing relatively inert proteins (milk or serum albumin) are added to the assay. However, the unspecific binding resulting in a background signal is not eliminated, thereby resulting in lower sensitivity of the assay.

A more recent method, immuno-PCR (e.g. U.S. Pat. No. 5,665,539), combines detection with an antibody and the polymerase chain reaction (PCR) to increase sensitivity for the detection of a specific protein. In a standard immuno-PCR protocol, an antibody attached to a nucleic acid sequence binds to an epitope on an antigen molecule. The attachment between the antibody and nucleic acid occurs via a linker with bispecific affinity for nucleic acids and antibodies, thus resulting in the formation of a specific antigen-antibody-DNA conjugate. Subsequently, a segment of the attached nucleic acid sequence is amplified by PCR and the PCR products are detected by gel electrophoresis.

However, the immuno-PCR method also has a relatively high background signal, resulting in lower sensitivity for the assay. One problem is that background levels are caused by non-specific binding of the nucleic acid template to the ELISA plastic wells. Although nucleic acids, such as salmon sperm DNA, are added to the reaction mixtures as part of the method in order to reduce nonspecific binding, the background is not eliminated. Also, the wells contain antibodies and often poorly defined components of the blocking buffer. These proteins and/or nucleic acids may themselves attach to the template, thus resulting in stearic hindrance and inaccessibility of the template for an enzyme. Furthermore, they may contain contaminating enzymatic activities, such as nuclease or polymerase activity, that are able to modify the template.

An alternative approach to detecting a substance of interest relies, like immuno-PCR, on linking of two molecules with defined activities. Such an approach is disclosed in U.S. Pat. No. 6,770,439. In that system, electrophoretic tag probes are used for the detection of target compounds. Each probe contains a target-binding moiety specific for a target compound, a cleavable linkage, a detection group, and a mobility modifier. When the linkage is cleaved, the mobility modifier is produced with a distinct charge/mass ratio and forms a distinct peak upon electrophoretic separation. As in the immuno-PCR protocol described above, this system does not link an antibody with an enzyme.

Antibodies linked to enzymes are widely employed as a diagnostic tool in medicine and plant pathology. They are also used as a quality control check in various industries, in particular in the food industry for detecting potential food allergens such as milk, peanuts, walnuts, almonds, and eggs. Antibodies tethered to enzymes are also widely used in biological research. As described in the example above, ELISA assays commonly have antibodies labeled with horseradish peroxidase or other enzymes, such as alkaline phosphatase.

For example, U.S. Pat. No. 6,610,479 discloses a polymerase that is non-covalently bound to antibody-coated beads in its inactive state. When heated, the polymerase separates from the antibody to form an active state. The polymerase can then be used in a PCR amplification reaction. However, heat must be added in such a system to render the polymerase active. In addition, background levels may be present from any bound polymerase-antibody complexes that are capable of PCR amplification because all of the steps occur in the same well.

In a different approach for providing a sensitive immuno-PCR method, Fischer et al. (J. Mol. Med. 85:461-469, 2007) devised a scheme whereby an antibody that is specific for a substance of interest (in Fischer's case, specific for an antibody that binds to an antigen of interest) is covalently linked to a reporter DNA. After binding of the antibody-DNA complex to the substance of interest, the DNA is cleaved from the antibody using a restriction endonuclease, and the cleaved DNA is subsequently used as a template in a PCR reaction. While the method of Fischer is suitable under some situations, it is limited in its application to immuno-PCR reactions and use of specifically designed nucleic acid templates having defined sequences.

Detection of a biological molecule by an antibody is a widely used method in biological research. Likewise, coupling the specificity of immunological detection with the sensitivity of PCR is also practiced in the art. However, the present inventors have realized that there still exists a need in the art for methods that allow detection of substances in a highly specific manner without an associated significantly high background level, which can obscure results and lower the sensitivity of a method.

### SUMMARY OF THE INVENTION

The present invention provides a new system for detection of a substance of interest. The system includes the use of a complex comprising a moiety, region, or portion that specifically binds to the substance of interest. The binding portion is linked to a second moiety, region, or portion, which has a catalytic activity that is capable of providing a detectable signal. The two portions are bound in a manner such that they can be released from one another by cleavage of at least one bond, such as a covalent bond, located in a linker portion linking the binding portion to the catalytic portion. In exemplary embodiments, the binding portion is an antibody or active portion thereof, and the catalytic portion is an enzyme or catalytic portion thereof. In embodiments, the linking portion comprises a peptide, polypeptide, or protein, or comprises a polynucleotide, preferably having a synthetic or engineered cleavage site for cleavage and separation of the binding region from the catalytic region. In preferred embodiments, the binding and catalytic portions (e.g., antibody and enzyme) are linked by way of a linking portion, which is cleaved by way of breaking of at least one covalent bond, to release the two portions from the complex. In general, the system combines the specificity of specific binding pair binding reactions (e.g., immunodetection) with the sensitivity of catalytic reactions (e.g., enzyme-based detection systems). It further allows for a reduction of background levels over systems that are commercially available by allowing for a two-step binding/detection process that reduces or eliminates various sources of background seen in commercially available systems.

In one aspect, the invention provides a detection reagent for detecting a substance of interest. In general, the detection reagent is a complex, typically a protein-containing complex, comprising a binding portion (e.g., an antibody or portion thereof) and a catalytic portion that comprises a catalytic activity (e.g., an enzyme portion), where the catalytic activity is a detectable activity. The binding and detection portions of the detection reagent are linked by a linker that is capable of cleavage in a specific or semi-specific manner to release the detection portion from its bonding to the binding portion. Preferably, the linker comprises a synthetic or engineered cleavage site that is designed to be a target site for cleavage by a pre-selected cleavage agent. As detailed below, the binding portion can be an antibody, which can be any antibody that specifically binds to a substance of interest, while the catalytic portion can be a catalyst, such as an enzyme, which may be any catalyst that can be used in an assay that produces, either directly or ultimately, a detectable signal. According to this aspect of the invention, use of a complex as a detection reagent is provided, where the complex comprises a binding portion, a linker portion, and a catalytic portion, where the binding portion and catalytic portion can be separated via specific cleavage, particularly at an engineered cleavage site.

In another aspect, the invention provides compositions comprising the detection reagent of the invention. In general, the compositions comprise the detection reagent and at least one other substance that is suitable for use in conjunction with the reagent. Suitable substances include those that may be caused to contact the reagent without adversely affecting its ability to perform as desired in a method according to the invention. Alternatively or in addition, compositions may comprise the detection reagent of the invention and one or more substances to which the reagent specifically binds (e.g., an antigen to be detected) or one or more substances that specifically or semi-specifically binds to the reagent (e.g., an enzyme or other molecule that cleaves the reagent at the linker region). Compositions of the invention may be found in liquid or solid form, such as, for example, in a lyophilized dried powder or in an aqueous mixture. Use of the composition in a binding and detection assay is accordingly provided.

In an additional aspect, methods of detection of substances of interest are provided. In general, the methods comprise: in a binding reaction vessel, causing a detection reagent of the invention to contact a sample containing or suspected of containing a substance of interest under conditions where the reagent can specifically contact the substance of interest, if present; removing unbound reagent; causing at least one bond linking the binding portion (or a portion thereof having binding activity) and the catalytic region (or a portion thereof having catalytic activity) of the detection reagent to break; removing the catalytic portion of the reagent from the binding reaction vessel and into a detection vessel; and causing the catalytic portion to catalyze a reaction that results, either directly or ultimately, in production of a detectable signal. As used herein, a binding reaction vessel is a container that contains substances for a binding reaction and where a binding reaction takes place. Similarly, a detection vessel is a container that contains substances for a detection reaction and where a detection reaction takes place. While not limited to any particular materials, shapes, or sizes, reaction vessels can be plastic microtiter plate wells or other containers known for use in specific binding pair binding reactions (e.g., antibody-antigen binding). Likewise, while not limited to any particular materials, shapes, or sizes, detection vessels can be plastic microfuge tubes, such as those suitable for PCR (e.g., QPCR). In a similar manner as mentioned above, this aspect provides for use of a detection reagent or composition of the invention in an assay for detecting the presence of a substance of interest in a sample.

In a further aspect, the invention provides kits comprising the detection reagent of the invention. In general, the kits comprise the detection reagent in at least one container, typically in combination with packaging materials for storage and/or shipment of the container. Although the detection reagent may be present in any amounts in the kits, typically the detection reagent is present in an amount that is adequate to practice a method according to the invention at least one time. In embodiments, multiple containers containing detection reagent are supplied in a kit. The detection reagent may be supplied as a pure substance or as part of a composition. Additional components may be supplied in the kit, including, but not limited to, other reagents and supplies for practicing a method of the invention. In view of this aspect, the invention provides for use of a kit for detection of a substance of interest.

### BRIEF DESCRIPTION OF THE DRAWINGS

The accompanying drawings, which are incorporated in and constitute a part of this specification, illustrate embodiments of the invention, and together with the written description, serve to explain certain principles of the invention.

FIG. 1 shows a generalized drawing of a detection reagent according to an embodiment of the invention. FIG. 1A depicts a reagent in its complete complexed form. FIG. 1B depicts the reagent after covalent cleavage of the complex at the linker portion. FIG. 1C depicts a particular embodiment of a detection reagent of the invention, comprising streptavidin as a binding portion, a peptide linker comprising a specific protease cleavage site, and Klenow polymerase as a catalytic portion.

FIG. 2 depicts general concepts of the method of the invention according to certain embodiments.

FIG. 3 generally depicts an exemplary chemistry for providing a covalent link between a binding portion and a catalytic portion, according to one embodiment of the invention. FIG. 3A depicts a thioether compound for linking a protein moiety of a binding portion to a nucleic acid tether or linker. FIG. 3B depicts a protein gel showing chemical coupling of an oligonucleotide to a streptavidin-maleimide molecule.

FIG. 4 depicts a method for creating a linker portion that can be covalently cleaved to release a binding portion from a catalytic portion according to one embodiment of the invention.

FIG. 5 depicts a method for linking an enzyme (catalytic portion) to a nucleic acid linker portion according to an embodiment of the invention. FIG. 5A shows the process for covalently attaching a molecule labeled with the benzylguanine (BG) substrate to any recombinant protein of choice. In this example, an oligonucleotide was labeled with BG then covalently coupled to recombinant Protein A-SNAP (Protein A binds IgG). FIG. 5B shows a silver stained acrylamide gel that demonstrates the SNAP tag coupling of an 80mer oligonucleotide (25 kD) to recombinant Protein A-SNAP.

### DETAILED DESCRIPTION OF VARIOUS EMBODIMENTS OF THE INVENTION

Reference will now be made in detail to various exemplary embodiments of the invention, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

As discussed above, reagents and methods for immunodetection of substances of interest are known and commercially available. However, these reagents and methods suffer from significant drawbacks, most notably either relatively low sensitivity, poor signal-to-noise ratio (high background signal), or both. In addition, some are specifically designed to function in only a certain manner, and are thus not adaptable for use broadly to detect substances of interest. While immuno-PCR reactions have been devised to improve sensitivity, commercially available technologies still suffer from high signal-to-noise ratio, limiting their usefulness. The present inventors have recognized that one way to improve signal-to-noise ratio while simultaneously retaining or improving specificity and/or sensitivity of detection assays is to provide a reagent that comprises a highly specific binding portion (e.g., an antibody portion) bound to a highly sensitive catalytic portion (e.g., an enzyme portion), where the reagent is designed such that the two portions may be separated from each other by cleavage of a bond, such as a covalent bond, in a linker portion between the two active portions. In this way, the detection reaction may be physically separated from the binding reaction, and background signal due to non-specifically bound reagent can be reduced or eliminated, while sensitivity retained or improved. Likewise, in embodiments, the reagent comprises a binding portion and a catalytic portion, where both portions are active and do not require or undergo inactivation and/or reactivation to provide their intended functionality in a binding and detection scheme.

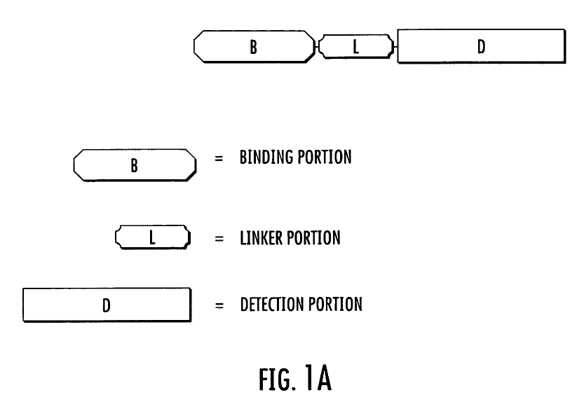

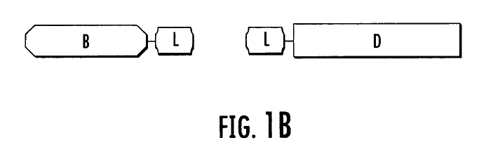

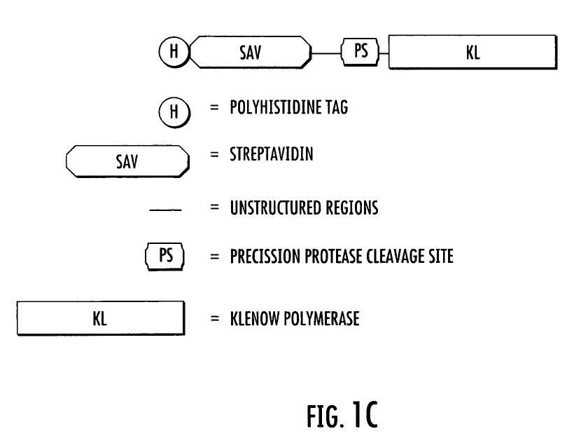

In achieving the present invention, the inventors developed a binding and detection reagent (referred to herein generally as a “detection reagent”). The reagent is capable of binding to a substance of interest in a sample and providing a detectable signal that is indicative of binding of the reagent to the substance. Broadly speaking, the binding and detection reagent comprises a binding portion, a catalytic portion, and a linker portion. The binding portion is capable of specifically binding to a substance of interest. The catalytic portion comprises an enzyme or other catalyst that is capable of catalyzing a reaction that, either directly or ultimately, produces a signal that can be detected. The linker portion serves to link the binding portion to the catalytic portion. The linker portion is designed such that it can be cleaved by a pre-selected cleavage reagent to release the catalytic portion from the binding portion. It thus comprises at least one site that has been engineered into the molecule as a site of cleavage. In exemplary embodiments, cleavage is by way of breaking one or more covalent bonds at the linker portion. The detection reagent of the invention is generally depicted in FIG. 1A, which shows the binding portion (B), linker portion (L), and catalytic portion (D). FIG. 1B generally depicts the binding and detection reagent after cleavage of the covalent linker portion. As will be immediately evident, release of the catalytic portion from the binding portion allows removal of the catalytic portion from the binding reaction environment, thus allowing for removal from possible contaminating materials. As will also be immediately apparent from the figure, the cleavage site can be placed anywhere within the linker portion. During preparation of the detection reagent, the cleavage site may be engineered or otherwise located at or near the fusion point of either or both of the binding portion or the catalytic portion with the linker portion. In such a situation, upon combining of the linker portion with the binding portion or the catalytic portion, the cleavage site is to be considered as part of the linker portion, even though it derives from a molecule generated separately and/or independently from the linker portion, per se. According to embodiments of the invention, detection of non-specific binding through the action of the catalytic domains will not occur because the catalytic domain will remain bound (non-specifically) to the original binding assay vessel, and will not be transferred to the detection vessel.

The binding portion (also referred to herein at times as the binding region) can be any substance that specifically binds to a substance of interest. In general, the binding portion comprises a member of a specific binding pair, which is capable, in its complexed form with the catalytic portion, of specifically binding to its complementary binding pair member. Numerous binding pairs are known and used in the art for linking of two things. Among the most widely used are antibody-antigen pairs, enzyme-substrate pairs, protein-protein complexes, and protein-small molecule complexes (e.g., p53/HIV-1 Tat; streptavidin/biotin). While any suitable specific binding pair may be used, in preferred embodiments, the binding portion comprises an antibody (or portion thereof), which specifically binds to an antigen of interest. In general, antibodies and their derivatives have advantageous specificity and avidity for their binding pair members, and are thus a preferred molecule for use as, or as part of, the binding portion. Those of skill in the art are well aware of how to make and use antibodies and fragments of antibodies (including both polyclonal and monoclonal, chimeric, humanized, etc.) for binding to antigens of interest. Accordingly, specifics about these molecules, their production, and their use, need not be detailed herein. It is sufficient to note that the antibody or antibody fragment should contain a specific binding region that is accessible for binding to its specific pair complement under conditions in which it is used, and is thus not sterically hindered or improperly folded when present as part of a multi-component complex. Again, those of skill in the art are fully aware of the portions of antibodies to be conserved for this purpose, and the manner in which such molecules should be conjugated to other molecules without interfering with activity. Likewise, enzymes known for commercial applications in detection assays are well characterized and suitable modifications, including deletions of portions of the enzymes, can be made without resort to trial-and-error experimentation. Applicants note that other molecules that serve as specific binding pair members are also known and characterized such that they can similarly be used in multi-component complexes without extensive trial-and-error experimentation to retain functionality.

It is to be understood that the substance of interest to be detected may be any substance of interest to a practitioner of the invention. It thus may be a naturally occurring substance or a manmade substance. It can be inorganic or organic in nature and may be present in any amount in a sample or reaction vessel. It thus may be present as a pure substance, as a minor component of a complex mixture of substances, or any level of purity in between. Among the many substances that can be of interest to a practitioner, mention may be made of the following non-limiting examples: biologically active substances, such as drugs (legal and illegal), hormones, proteins (from any source), substances of bacterial or viral origin (e.g., lipopolysaccharides, toxins, viral proteins), and substances indicative of a particular biological state (e.g., cancer-specific antigens). In general, any substance that is known to be detectable using immunological or other assays using specific binding pair members may be a substance according to the present invention.

The binding portion of the reagent of the invention may comprise multiple sub-portions, each having the same or different activities. Thus, for example, a binding portion may comprise two antibodies or parts thereof. Each of the antibodies or portions thereof can be the same or different (e.g., both antibodies; one an antibody, one a portion of an antibody, etc.), and each can be specific for the same substance or one can be specific for a first substance while the other is specific for a second substance, etc. Furthermore, the binding portion may comprise, in addition to the molecule that shows specificity for the substance of interest, one or more additional molecules, the only restriction being that the additional molecule(s) do not interfere with the activity of the molecule with specificity for the substance of interest. Thus, for example, a binding portion may comprise an antibody bound to a molecule that is capable of conjugating the antibody to additional molecules. For example, an antibody may comprise a biotin tag that allows for conjugation of additional molecules to the antibody. In an exemplary embodiment, an antibody is covalently bound to biotin, which allows for conjugation of a linker portion and a detection portion to the antibody by way of a streptavidin-biotin interaction. Likewise, the binding portion may comprise one or more molecules that allow for detection of the binding portion. In this way, reactions may be performed to follow binding of the binding portion to the substance of interest and, possibly, other substances.

The binding portion may comprise two moieties, each of which can be considered as a member of a specific binding pair. For example, a binding portion may comprise on one end an antibody that is specific for a substance of interest (e.g., an antigen, another antibody, etc.), and on the other end, a streptavidin molecule that allows for linking of the antibody to another portion of the binding region by way of binding to biotin. Another non-limiting example of a bi-functional binding region is one that includes on one end an antibody or portion thereof and on the other end a single-stranded nucleic acid. The antibody may be specific for an analyte of interest, while the single-stranded nucleic acid may have a known sequence that can hybridize (under any pre-defined conditions) to a complementary strand. Any number and permutation of binding members may be including in the binding portion to create a functional binding portion for a given purpose.

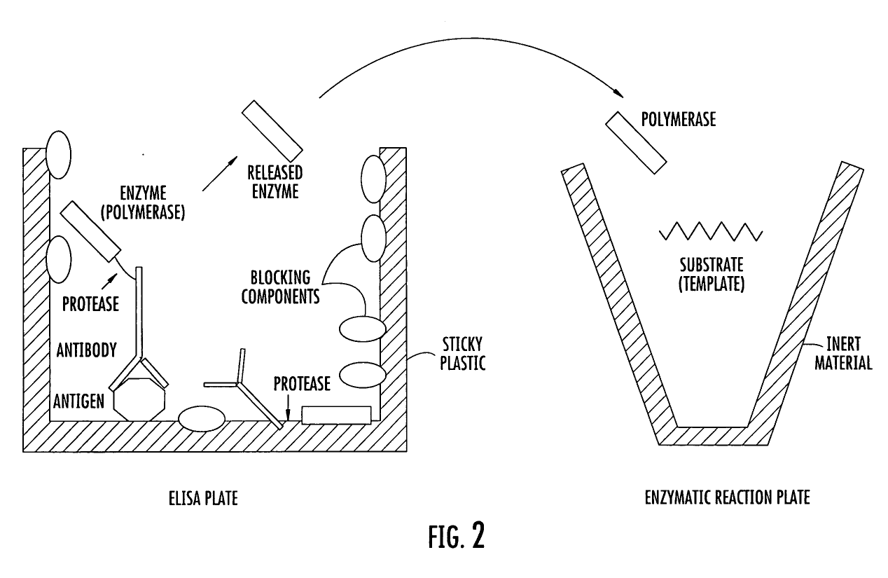

The detection reagent of the invention further comprises a linker portion, which generally serves to link the binding portion to the catalytic portion (see, for example, FIGS. 1 and 2). The linker portion may comprise any suitable chemical entity. In exemplary embodiments, it comprises a polyamino acid or a polynucleic acid. The linker portion contains at least one site that can be cleaved to break a bond, resulting in separation of the binding portion from the detection portion. In preferred embodiments, the bond that is cleaved is a covalent bond. Cleavage may be in a specific or semi-specific manner. As used herein, specific cleavage is cleavage that can not only be predicted to occur at a specific site, but that will occur at a site that is either unique within the reagent or that is unique within the solvent-exposed portions of the reagent. In contrast, as used herein, semi-specific cleavage is cleavage that occurs at a pre-defined specific site (like specific cleavage). However, semi-specific cleavage allows for the pre-defined specific site to be present on the reagent in two or more places, which may or may not be solvent accessible. Typically, such sites are all present within the linker portion of the molecule. For example, a binding portion comprising a proteinaceous active moiety may be linked to a catalytic portion comprising a proteinaceous active moiety by way of a nucleic acid linker. The nucleic acid linker may comprise multiple binding sites for a dye that causes covalent cleavage of the nucleic acid when exposed to ultraviolet (UV) irradiation. Such sites are semi-specific according to the invention. In contrast, the same configuration would show specific cleavage if a known restriction endonuclease site were to be engineered into the nucleic acid linker. Non-limiting examples of types of associations susceptible to cleavage at a linker region include: calmodulin and calmodulin-binding peptide portions, the interaction being destroyed by EGTA; a polyhistidine tag and a nitroacetic acid group, the interaction being destroyed by imidazol or EGTA; glutathione-S-transferase and glutathione, the interaction being destroyed by excess of reduced glutathione; and two partially complementary oligonucleotides that are holding a linker region together by hydrogen bonding, the interaction being destroyed by an invading oligonucleotide that disrupts the hydrogen bonding. Likewise, another non-limiting example is any reagent or set of reagents that find use in affinity purification processes.

The linker portion may be comprised of any substance or combination of substances. Typically, however, it will be comprised of proteinaceous material or nucleic acid material. Owing to the fact that amino acid sequences and nucleic acid sequences can easily be engineered to contain specific cleavage sites, and owing to the fact that the chemistries for linkage of proteins (including peptides and polypeptides) and nucleic acids to proteins are well known and easily practiced, linker portions comprising amino acid sequences and nucleotide sequences, and in particular amino acid sequences, are preferred. There are numerous specific and semi-specific amino acid sequences known in the art that may be used for cleavage, including covalent cleavage, and any one of such sequences may be used.

Exemplary nucleases include: AaII, AarI, AasI, AatII, Acc65I, AccB7I, AccI, AccIII, AciI, AclI, AcuI, AdeI, AfeI, AflII, AflIII, AgeI, AhdI, AleI, AloI, AluI, Alw21I, Alw26I, Alw44I, AlwI, AlwNI, ApaI, ApaLI, ApeKI, ApoI, AscI, AseI, AsiSI, AvaI, AvaII, AvrII, BaeI, BalI, BamHI, BanI, BanII, BbsI, BbuI, BbvCI, BbvI, BccI, BceAI, BcgI, BciVI, BclI, BcnI, BcuI, BfaI, BfiI, BfmI BfrBI, BfuAI, BfuCI, BfuI, BglI, BglII, BlpI, Bme1390I, Bme1580I, BmgBI, BmrI, BmtI, BoxI, BpiI, BplI, BpmI, Bpu10I, Bpu1102I, BpuEI, BsaAI, BsaBI, BsaHI, BsaI, BsaJI, BsaMI, BsaWI, BsaXI, BseDI, BseGI, BseJI, BseLI, BseMI, BseMII, BseNI, BseRI, BseSI, BseXI, BseYI, BsgI, Bsh1236I, Bsh1285I, BshNI, BshTI, BsiEI, BsiHKAI, BsiWI, BslI, BsmAI, BsmBI, BsmFI, BsmI, BsoBI, Bsp19I, Bsp120I, Bsp1286I, Bsp1407I, Bsp143I, Bsp143II, Bsp68I, BspCNI, BspDI, BspEI, BspHI, BspLI, BspMI, BspPI, BspQI, BspTI, BsrBI, BsrDI, BsrFI, BsrGI, BsrI, BsrSI, BssHII, BssKI, BssSI, Bst1107I, Bst98I, BstAPI, BstBI, BstEII, BstF5I, BstNI, BstOI, BstUI, BstXI, BstYI, BstZI, BstZ17I, Bsu15I, Bsu36I, BsuRI, BtgI, BtgZI, BtsCI, BtsI, BveI, Cac8I, CaiI, CfoI, Cfr10I, Cfr13I, Cfr42I, Cfr9I, CfrI, ClaI, CpoI, Csp45I, Csp6I, CspI, CspCI, CviAII, CviKI-1, CviQI, DdeI, DpnI, DpnII, DraI, DraIII, DrdI, EaeI, EagI, Eam1104I, Eam1105I, EarI, EciI, Ecl136II, EclHKI, Eco105I, Eco130I, Eco147I, Eco24I, Eco31I, Eco32I, Eco47I, Eco47III, Eco52I, Eco57I, Eco57MI, Eco72I, Eco81I, Eco88I, Eco91I, EcolCRI, EcoNI, EcoO109I, EcoP15I, EcoRI, EcoRV, EheI, Esp3I, FatI, FauI, Fnu4HI, FokI, FseI, FspI, FspAI, GsuI, HaeII, HaeIII, HgaI, HhaI, Hin1I, Hin4I, Hin6I, HincII, HindIII, HinfI, HinP1I, HpaI, HpaII, HphI, Hpy166II, Hpy188I, Hpy188III, Hpy8I, Hpy99I, HpyAV, HpyCH4III, HpyCH4IV, HpyCH4V, HpyF10VI, Hsp92I, Hsp92II, I-PpoI, KasI, Kpn2I, KpnI, KspAI, LweI, MbiI, MboI, MboII, MfeI, MisI, MluI, MlyI, MmeI, MnlI, Mph1103I, MscI, MseI, MslI, MspA1I, MspI, MssI, MunI, Mva1269I, MvaI, MwoI, NaeI, NarI, NciI, NcoI, NdeI, NdeII, NgoMIV, NheI, NheI-HF, NlaIII, NlaIV, NmeAIII, NmuCI, NotI, NruI, NsbI, NsiI, NspI, OliI, PacI, PaeI, PaeR7I, PagI, PauI, PciI, PdiI, PdmI, Pfl23II, PflFI, PflMI, PfoI, PhoI, PleI, PmeI, PmlI, PpiI, PpuMI, PshAI, PsiI, Psp1406I, Psp5II, PspGI, PspOMI, PspXI, PstI, PsuI, PsyI, PvuI, PvuII, PvuII-HF, RsaI, RsrII, SacI, SacII, SalI, SalI-HF, SapI, SatI, Sau3AI, Sau96I, SbfI, ScaI, ScaI-HF, SchI, ScrFI, SdaI, SduI, SexAI, SfaNI, SfcI, SfiI, SfoI, SgfI, SgrAI, SinI, SmaI, SmiI, SmlI, SmuI, SnaBI, SpeI, SphI, SphI-HF, SspI, StuI, StyD4I, StyI, SwaI, TaaI, TaiI, Taq^{α}I, TaqI, TasI, TatI, TauI, TfiI, TliI, Tru1I, Tru91, TseI, Tsp45I, Tsp509I, TspMI, TspRI, Tth111I, TurboNaeI, TurboNarI, Van91I, VspI, XagI, XapI, XbaI, XceI, XcmI, XhoI, XhoII, XmaI, XmaJI, XmiI, XmnI, and ZraI. The corresponding cleavage sites for these enzymes are known in the art.

Additional non-limiting examples of specific nucleases include the following. 1) BAL 31, which can cleave internally in double-stranded DNA molecules that contain helical distortions. 2) homing endonucleases, which are double stranded DNases that have large, asymmetric recognition sites (12-40 base pairs) and coding sequences that are usually embedded in either introns or inteins. Homing endonuclease recognition sites are extremely rare. For example, an 18 base pair recognition sequence will occur only once in every 7×10¹⁰ base pairs of random sequence. This frequency is equivalent to only one site in 20 mammalian-sized genomes. It is to be noted that homing endonucleases tolerate some sequence degeneracy within their recognition sequence, and thus, the functional sequence specificity may be as low as 10-12 base pairs. Accordingly, homing endonucleases may be considered either specific or semi-specific in their mode of action. Examples of homing endonucleases include I-CeuI, I-SceI, PI-PspI, and PI-SceI. 3) Nicking endonucleases and similar functioning nucleases, such as Nuclease S1. Nicking endonucleases and similar acting enzymes hydrolyze only one strand of duplex DNA to produce nicked, rather than cleaved, molecules. The nicked DNA may be completely hydrolyzed by an additional agent. Exemplary nicking enzymes include Nt.BstNBI, Nb.BsrDI, Nb.BtsI, Nt.AlwI, Nb.BbvCI, and Nt.BbvCI.

Among semi-specific nucleic acid cleavage means, mention may be made of not only combinations of small molecules that can cause DNA cleavage (e.g., ternary Cu(II) complexes containing histamine and amino acids; Reddy et al., Tetrahedron Letters 47(41):7311-7315, 2006) and intercalating agents and the like that can be activated by exposure to energy to break covalent nucleic acid bonds (e.g., ethidium bromide, piperidine and other amines), but enzymatic semi-specific means as well. For example, the Type IC restriction endonuclease reported by Szczelkun et al. (J. Mol. Biol. 271(1): 112-123(12), 1997) may be used. As disclosed by Szczelkun et al., the Type IC restriction endonuclease Eco R124I binds specifically to its recognition sequence but subsequently translocates non-specific DNA past the complex in an ATP-dependent mechanism. The enzyme, and enzymes of this class, thus have the potential to cleave DNA at loci distant from the recognition site. In addition, exemplary enzymatic semi-specific cleavage agents, sometimes referred to as “scission reagents”, such as DNA-binding proteins linked to the chemical nuclease 1,10-phenanthroline-copper (Pan et al., Mol. Microbiol. 12(3):335-342, 1994), may be used.

Among exemplary specific and semi-specific enzymatic cleavage reagents for proteins, mention may be made of: Trypsin, Endoproteinase, Clostripain, Papain, Armillaria mellea protein, Staphylococcus aureus protein, Endoproteinase, Asparaginylendopeptidase, Chymotrypsin, Thermolysin, Pepsin, Subtilisin, Elastase, Thrombin, and Bromelain. Mention may also be made to chemical reagents that semi-specifically cleave amino acid bonds, such as, but not limited to, cyanogen bromide, dilute acid (pH about 2), formic acid (about 80%), hydrochloric acid (about 6 M), hydroxylamine, DMSO/HCl/HBr, and NTCB.

Other exemplary reagents for specific and semi-specific cleavage of linker portions are disclosed herein or will be immediately apparent to those of skill in the art. Those of skill in the art are thus aware of other exemplary specific and semi-specific nucleic acid and protein cleavage reagents, and accordingly all such reagents need not be named herein. The reagents specifically recited are exemplary only and are not exemplified because they are preferred over non-exemplified reagents in any way.

Further, as detailed in the Examples below, the present inventors have devised specific exemplary enzymatically cleavable linkers/spacers that separate a binding portion and a catalytic portion. However, according to the teachings of this invention, the cleavable linker portion does not have to be enzymatically cleavable; rather, it alternatively can be chemically cleavable. There are many methods known to those skilled in the art for designing cleavable linkers that contain chemically cleavable groups. By way of example, acid-cleavable linkers (van der Veken, P. et al., Chembiochem 6:2271-2280, 2005), diazobenzene linkers (Fonovic, M. et al., Mol. Cell. Proteomics 6:1761-1770), and disulfide-cleavable linkers (Shimkus, M. et al., Proc. Natl. Acad. Sci. U.S.A., 82:2593-2597, 1985) can be used for this purpose (each of which is incorporated herein by reference). For example, a disulfide-cleavable nucleotide (Shimkus, M. et al., Proc. Natl. Acad. Sci. U.S.A. 82:2593-2597, 1985) can be introduced into a polynucleotide linker, and the polynucleotide linker can be used to conjugate a binding portion and a catalytic portion. Next, the conjugate can be used for detection of analytes essentially as described in Example 4 (below), except the cleaving between the binding, tethering, and catalytic portions is achieved by applying a reducing agent, for example 50 mM dithiothreitol, rather than a protease. The released catalytic portion can be transferred into a detection vessel (e.g., a separate tube or a plate) where a detection reaction is performed. As will be understood by those of skill in the art, the latter is contingent upon the nature of the catalytic portion.

The linker portion may be provided to the complex as a preformed moiety, which is linked to the catalytic portion and the binding portion by way of known linking, either in a single reaction or a two-step process. Alternatively, however, the linker portion may be provided to the complex as a portion of one or both of the binding portion and the catalytic portion. For example, a binding portion of a complex may contain an antibody linked to a single-stranded nucleic acid, while a catalytic portion may contain an enzyme linked to a single-stranded nucleic acid, where the two nucleic acids are complementary over at least a portion of their sequences and thus can hybridize to each other under pre-selected conditions (e.g., high stringency, moderate stringency, low stringency). For example, the nucleic acids can show a complementarity level over one or both of their sequences of 50% or greater, such as 60% or greater, 70% or greater, 80% or greater, 90% or greater, 95% or greater, 98% or greater, 99% or greater, or 100%. In such embodiments, the linker portion can be defined in terms of function rather than structure (e.g., the region where cleavage occurs between the binding portion and the catalytic portion).

The detection reagent may be provided as a pure substance or as part of a composition. In general, the compositions comprise the detection reagent and at least one other substance that is not harmful or deleterious to the structural integrity or functionality of the reagent. The compositions of the invention are thus not particularly limited in their make-up. Exemplary compositions of the invention include stock solutions comprising concentrated detection reagent for use in methods according to the invention. They further include reaction mixtures for use in binding of the detection reagent to one or more substances of interest (e.g., mixtures including samples suspected of comprising a substance of interest) and reaction mixtures for use in cleavage reactions to separate the binding portions of the detection reagent from the catalytic portions. Compositions can comprise one or more liquids, solids, or combinations, including, but not limited to aqueous mixtures, such as biochemical reaction mixtures or buffers. In some embodiments, compositions comprise one or more substances that are inhibitory to an activity of the reagent; however, when diluted to a working concentration, the amount of inhibitory substance is reduced to a level that is not inhibitory.

Exemplary compositions according to the invention comprise the detection reagent of the invention and one or more of the following substances: a substance that can be specifically bound by the binding portion of the detection reagent; a substance that can participate in cleavage of a bond in a linker portion of the detection reagent; a solid support to which the binding reagent, the substance of interest to be detected, or another substance can bind; and a substance that can reduce non-specific binding of the binding reagent to a solid support. It is to be understood that the term “solid support” means any substance that can be used to bind other substances and is insoluble under a given condition. Typically, a solid support is a bead, membrane, vessel (e.g., reaction vessel or reaction vessel wall) made from solids or mixtures of solids. Exemplary solid supports include: beads, such as those made from polymeric materials (e.g., latex, nylon, plastic, and other natural or manmade compounds); membranes, such as those made from polymeric materials; and reaction vessels, such as microtiter plates, microfuge tubes, test tubes, which may be made from any suitable materials known in the art. In some embodiments, the solid supports are capable of binding the substance of interest to be detected, whereas in other embodiments, the substance is not reactive with respect to the solid substrate. Likewise, the solid substrate may be reactive with the detection reagent of the invention or one or more other substances that are used in a method according to the invention.

In an exemplary embodiment, a solid support is used, where the solid support has bound to it an antibody that is capable of detecting a substance of interest. In such an embodiment, detection of the substance of interest can be by way of a “sandwich” assay, in which an antibody is bound to the solid support, the substance of interest is bound to the antibody, and the substance is further bound by a detection reagent of the invention. In some embodiments, a solid support is made from materials that do not bind to the detection reagent or portions thereof to any appreciable extent.

The reagents and compositions of the invention can be used in methods of detecting a substance of interest. In general, the methods of detecting comprise: causing a detection reagent of the invention to contact a sample containing or suspected of containing a substance of interest under conditions where the reagent can specifically contact the substance of interest, if present; removing unbound reagent, non-specifically bound reagent, or both; causing a bond linking the binding and catalytic portions of the reagent to break; physically separating the catalytic portion of the reagent from the binding portion; and causing the catalytic portion to catalyze a reaction that results, either directly or ultimately, in production of a detectable signal. In embodiments, the detection reagent comprises an antibody binding portion and an enzyme catalytic portion. In embodiments, the binding portion is covalently linked to the catalytic portion by a nucleic acid or protein linker portion. A general depiction of an embodiment of the method of the invention is shown in FIG. 2.

According to the method, the act of contacting or causing a thing to contact another thing is any action that results in physical contact between the two things mentioned. Thus, it can be combining two compositions in a manner that allows for mixing and contact of a reagent with a substance of interest. Likewise, it can comprise substantial physical intervention, such as by mixing (e.g., swirling, pipetting, vortexing), causing a fluid to flow across a solid support (e.g., filtering), and the like.

The term “sample” is used broadly herein to denote any substance or any composition that contains, or is suspected of containing, a substance of interest. A sample thus may be one taken from a particular environment (e.g., tissue sample, outdoor environmental sample), or one created by combining two or more substances, compositions, etc. It thus may be taken from a patient (human or animal) or may be isolated from an environmental site of interest (e.g., drinking water supply, landfill, farmland). The sample can contain any type of substance of interest, such as both harmful and beneficial molecules and compounds. Included among the substances are: poisons, toxins, man-made chemicals, organisms (e.g., bacteria, viruses), proteins and nucleic acids and other molecules produced by living organisms, and products of metabolic activity in living organisms. While the sample may be of any type, typically it is an aqueous composition in which the reagent of the invention is capable of movement, for example by simple diffusion or Brownian movement, and contact with a substance of interest, if present. It is to be noted that the substance of interest need not be present in the sample at any particular concentration or amount; however, the ability of the method to detect it will relate to the concentration/amount present and the particular identity of the binding and catalytic portions.

In general, the method is practiced in at least one reaction vessel capable of holding a suitable amount of reaction volume. For example, the method may be practiced in a well of a microtiter plate, such as one commercially available made from plastic and having 96 wells. Likewise, it may be practiced in a microfuge tube, such as one designed or suitable for performing a PCR reaction or other enzymatic reaction. In preferred embodiments, the reaction vessel is one onto the surface of which one can bind a molecule for anchoring the substance of interest. For example, the reaction vessel may act as a solid support for attaching an antibody that specifically binds to the substance of interest. Many such solid supports/vessels are known in the art, as are methods for producing them. Accordingly, details of such solid supports and their fabrication need not be detailed herein.

The method of detection includes removing unbound or non-specifically bound reagent. Typically, to ensure binding to all or as much of the substance of interest as possible, the method will include adding an excess of detection reagent to a vessel in which the binding reaction takes place. The detection reagent is designed to specifically bind to a substance of interest; however, non-specific binding to other things, including the solid support, may also occur. Likewise, where excess reagent is added, unbound reagent might exist in the reaction mixture. The non-specifically bound and the unbound reagent can interfere with subsequent method steps. Therefore, the method of the invention comprises removing at least some of the unbound or non-specifically bound reagent. The act of removing may be any act that results in removal of some or all of the unbound or non-specifically bound reagent in the binding reaction vessel or on a solid support comprising specifically bound detection reagent. For example, it may be by way of pipeting away some or all of the liquid in the reaction vessel, by way of decanting some or all of the liquid in the reaction vessel, by washing a membrane or bead comprising specifically bound detection reagent, or any other action that removes unbound or non-specifically bound reagent. Where removal of non-specifically bound reagent is desired, it is preferable to use some level of physical disruption to dislodge the reagent. For example, one or more washing steps, in which an appropriate aqueous composition is added and removed from the reaction vessel can be used. As part of the washing, the washing composition may be caused to move about the reaction vessel to cause physical separation of non-specifically bound reagent (which will be bound with a lower avidity than specifically bound reagent). In embodiments, the washing composition is caused to swirl or otherwise become agitated in the reaction vessel to cause removal of some or all of unbound and non-specifically bound reagent.

The method of detection also comprises causing at least one covalent bond linking the binding and catalytic portions of the detection reagent to break. As discussed above, the binding reagent comprises a binding portion linked to a catalytic portion by way of a linker. In contrast to methods known in the art, the present invention links a binding portion to a catalytic detection portion by way of a linker that can be cleaved to provide separate binding and detection functionalities. In preferred embodiments, the linker comprises a cleavage site that has intentionally been engineered into the linker region to provide a cleavage site for a pre-selected cleavage reagent (e.g., a protease cleavage site is engineered into the linker region for later cleavage by a pre-selected protease specific for that cleavage site). The presence of a cleavable covalent bond provides many advantages. Among them, mention may be made of the fact that it allows stringent washing of the binding reaction to be performed, which can result in a reduction of background signal due to non-specifically bound reagent, as compared to other methods known in the art. It also allows for specific removal of the catalytic portion from the binding portion under controlled reaction conditions. This permits the catalytic domain to be removed from the surface region of the vessel. When the catalytic domain is held near the surface it must interact with the plastic and all the molecules adhered to the plastic. This can effect the efficiency of its reactivity. In embodiments, ideally the catalytic domains involved in desired immune complexes are cleaved (presumably they stick up more from the surface and are more accessible) while those that are non-specifically bound may be cleaved less efficiently (they are held closer to the surface and are less accessible to be cleaved). If true, then the non-specifically bound molecules will be retained in the original assay vessel even if non-specific binding is occurring through the non-catalytic domains.

The bond cleavage can be accomplished by any suitable method. Exemplary covalent bonds and specific and semi-specific cleavage reagents are discussed herein. Those of skill in the art can easily perform bond cleavage, including covalent bond cleavage, according to the present invention without undue or excessive experimentation because the cleavage reactions follow known chemical and biochemical principles that need not be altered substantially to function. In preferred embodiments, cleavage is specific cleavage of a polyamino acid linker sequence or specific cleavage of a polynucleotide linker sequence, such as through use of proteases and endonucleases, respectively.

The method of detecting a substance of interest comprises, after cleavage of a bond at the linker portion, physically separating the catalytic portion of the reagent from the binding portion. The act of separating may be any action that results in physical separation of most, preferable all or substantially all, of the catalytic portion from the binding portion and/or from intact detection reagent (e.g., detection reagent non-specifically bound to a solid support). It thus may comprise aspiration or decanting of soluble material from insoluble material, such as by removal of the liquid portion of the mixture by pipetting. The act of separating can include any action or series of actions, and thus may include use of one or more intrinsic physical or functional properties of the catalytic portion, binding portion, or linker portion. For example, separating can include centrifugation to pellet insoluble material from soluble material. It also may include binding of one portion to an affinity molecule, which is suitable for specific binding of the portion of interest (e.g., passing the reaction mixture over an affinity column to bind the catalytic portion, with subsequent release of the catalytic portion into a new reaction vessel). It will be immediately apparent to those of skill in the art that many separation techniques will be suitable, and that any of those techniques may be used within the context of the invention.

In exemplary embodiments, detection reagent is bound to a substance of interest that is, in some way, anchored to the surface of a solid support. In such a situation, the act of separating can comprise using physical means to separate the insoluble solid support from a soluble detection portion. There are numerous ways to achieve such separation, and all are contemplated by the present invention. Among the exemplary ways, mention may be made of simple aspiration (e.g., pipetting) or decanting of all or a portion of the liquid reaction mixture from the reaction vessel.

The method may further comprise introducing the separated catalytic portion into a second vessel, also referred to herein as a detection vessel. An advantage of the present invention over other solutions for detection of substances of interest is the separation of the binding reaction and the detection reaction. Separation of the binding portion from the catalytic portion, and performing a sensitive detection assay in the absence of substantial contamination by the binding portion, non-specifically bound detection reagent, and unbound detection reagent provides a high level of sensitivity. Sensitivity is achieved, at least in part, by removing the catalytic domain from the surface (plastic, blocking agents, non-specifically bound detection reagents) and allowing it to be put into solution in a reaction vessel with a reaction mixture for the detection step. The step of introducing can comprise any action that results in the presence of a catalytic portion according to the invention in a detection vessel. It thus may include pipetting of an aqueous composition into the detection vessel, pouring of an aqueous composition into the detection vessel, placing a dried (e.g., lyophilized) powder into the detection vessel, etc.

The method of detecting further comprises the step of causing the catalytic portion of the detection reagent to catalyze a reaction that results, either directly or ultimately, in production of a detectable signal. The catalytic reaction may be any catalytic reaction that can result in a detectable signal, and thus may be by way of a chemical catalyst or a biochemical catalyst (enzyme, including DNAzyme). There are numerous such catalytic reactions known in the art, and any such reaction is envisioned as part of the present invention. In exemplary embodiments, the catalytic reaction is one that produces a signal detectable as electromagnetic radiation, such as visible light or ultraviolet light. Many catalysts are known that can produce a product that generates light, either intrinsically or after being acted upon by a subsequent catalyst or energy donor. Fluorophores are one exemplary class of reaction products. In preferred embodiments, the catalyst is an enzyme that produces a detectable product. For example, the enzyme may be a polymerase that can generate a double-stranded nucleic acid from a template nucleic acid, where the polymerase is capable of incorporating a modified nucleotide that is detectable, or where the nucleic acid produced by the polymerase is detectable by exposure to a dye (e.g., ROX, SYBR Green).

As mentioned above, there are assays known in the art that include detection of a substance of interest using an antibody, followed by amplification of a nucleic acid tethered to the antibody. One recent improvement to those assays includes cleavage of the tethered nucleic acid, removal of the cleaved portion to a new reaction vessel, and PCR amplification of the cleaved nucleic acid. In contrast to the currently known technologies, the present invention includes a detection reagent that comprises a binding portion and a catalytic portion, where the catalytic portion has an assayable catalytic activity. The catalytic activity may be any activity chosen by the practitioner. For example, the catalytic portion may comprise horseradish peroxidase, which can be used to catalyze conversion of o-phenylenediamine dihydrochloride to produce a colored product. Alternatively for example, the catalytic portion may comprise alkaline phosphatase, which can be used to catalyze fluorescence from the fluorogenic substrate methylumbelliferyl phosphate (MUP) or catalyze light emission from the calorimetric substrate p-nitrophenyl phosphate (pNPP). Non-limiting exemplary catalytic regions comprise a nucleic acid polymerase, such as a DNA-dependent DNA polymerase, a DNA-dependent RNA polymerase, and an RNA-dependent DNA polymerase. For example, a catalytic region according to a reagent of the invention can comprise a polymerase, or catalytic portion thereof, suitable for use in an acellular nucleic acid amplification reaction, such as PCR and its various derivatives (e.g., RT-PCR, QPCR, QRT-PCR). Those of skill in the art are well aware of such amplification reactions and polymerases for use in them, and any such reaction and polymerase (or portion thereof) can be used according to the present invention. Thus, for example, the catalytic portion may comprise a thermostable or thermolabile polymerase from a eukaryote or a prokaryote (including both eubacteria and archaea), such as, but not limited to, Pyrococcus furiosus (Pfu) DNA polymerase, E. coli DNA polymerase I, T7 DNA polymerase, Thermus thermophilus (Tth) DNA polymerase, Bacillus stearothermophilus DNA polymerase, Thermococcus litoralis (Tli) DNA polymerase (also referred to as Vent DNA polymerase, 9° Nm DNA polymerase, Thermotoga maritima (Tma) DNA polymerase, Thermus aquaticus (Taq) DNA polymerase, Pyrococcus kodakaraensis (KOD) DNA polymerase, JDF-3 DNA polymerase, Pyrococcus GB-D (PGB-D) DNA polymerase, T3 DNA polymerase, T4 DNA polymerase, T5 DNA polymerase, T7 DNA polymerase, Klenow fragment, and Φ29 DNA polymerase. Numerous other catalytic reactions are known in the art and widely available. Thus, the detection reagents and methods of the invention are versatile and provide the practitioner with a wide range of options for designing assays to detect substances of interest.

Accordingly, the present invention is not limited to reagents and methods for immuno-PCR, but rather broadly captures the concept of specific binding to a substance of interest and subsequent sensitive detection by way of a catalytic reaction using a bi-functional detection reagent having both binding and catalytic activities. While the present invention can relate, in embodiments, to an immuno-PCR assay and reagents therefor, it is distinct in its design and operation from known immuno-PCR assays and reagents. For example, use of a bi-functional reagent according to the present invention is not only not contemplated in publicly known systems, but provides advantages over current publicly known reagents and systems that are not provided by the current publicly known reagents and systems. For example, in currently known immuno-PCR assays, an unknown amount of template nucleic acid is introduced into a PCR reaction mixture, and amplified product is detected. For small amounts of template nucleic acid, a lag period for detection of specific signal from amplification may be present, which may cause the PCR reaction to require many (e.g., greater than 30) cycles. In contrast, in embodiments of the present invention that relate to immuno-PCR, the assays allow for inclusion of a known amount of nucleic acid template, which may be relatively high. Detectable amplification upon introduction of the released detection reagent (i.e., polymerase) thus may begin within a very few cycles of the PCR reaction, resulting in shortened times for detection.

Likewise, a detection reagent of the invention may be designed such that it comprises a proteinaceous binding portion, a proteinaceous linker portion, and a proteinaceous catalytic portion. In such embodiments, the stability of each portion can be retained to a very high extent by selecting reaction conditions for binding and cleavage that are suitable for proteins (e.g., moderate salt concentrations, relatively cool temperatures, low levels of detergents), while at the same time, not taking into consideration the need for conditions that are preferable for nucleic acids (e.g., lack of nucleases or inclusion of inhibitors of nucleases, etc.). So, for example, a complex sample from a biological source, which is suspected of containing one or more nucleases, may be assayed directly using a reagent according to embodiments of the present invention, whereas the sample would require pre-treatment to reduce or eliminate nuclease activity if used in currently known immuno-PCR reactions. In addition, such a protein-protein-protein complex is easier and more reproducible to manufacture than a typical chemical protein conjugation of proteins and/or nucleic acids. Furthermore, with regard to all types of complexes of the invention, cleavage and use of the catalytic domain in a solution phase rather than when affixed to a solid surface achieves better mixing and substrate accessibility.

Of course, as with any process, method, assay, etc. one or more positive and/or negative control reactions may be performed at one or more of the steps. The control reactions can be designed to verify that a particular step or series of steps were performed as designed, and/or that materials and reagents used in the method functioned as expected. Those of skill in the art can devise suitable control reactions without the need for each to be specified herein.

The reagents and methods of the present invention have broad applicability in the biotechnology and medical fields. They are useful not only for research purposes, but are also advantageously applied to the medical and veterinarian arts as diagnostic reagents and assays. Thus, in embodiments, the present invention provides diagnostic methods for detecting substances of interest from a sample, where detection of the substances is indicative of particular diseases or disorders. For example, a method of the invention may be a diagnostic method for diagnosing cancer in a patient, where the method includes detection of a cancer-specific antigen. Likewise, for example, a method of the invention may be a diagnostic method for diagnosing a metabolic disorder, such as diabetes, by detection of a known marker of the disorder. Many markers for diseases and disorders in humans and other animals are known, and any such marker may be used within the context of a diagnostic assay according to the invention. It is to be noted that the marker need not be one that is 100% correlated with a particular disease or disorder, but rather may simply be known as associated with a disease or disorder in some humans or animals affected by the disease or disorder. Furthermore, it is to be understood that the marker need not be a causative agent of the disease or disorder, but rather may be any marker that is know to be associated (by its presence, absence, or level) with a disease or disorder.

Methods of the invention may also be used to prognose the outcome of a disease or disorder, or follow a treatment regimen for a disease or disorder. More specifically, the reagents and methods of the invention can be used to follow the presence and level of markers for diseases and disorders over time by repetition of detection assays on multiple samples taken over time. Where the presence, absence, or level of a marker is indicative of the likelihood of recovery from the disease or disorder (or the severity of the disease or disorder, etc.), the assays of the present invention can be used to predict the outcome or follow the development of the disease or disorder. This embodiment of the invention is highly relevant to situations where the person or animal is under a treatment regimen for the disease or disorder, and the marker can be used as an indicator of the success of the treatment regimen. It is also highly relevant to situations where a disease or disorder is being treated, and the treatment is known to be associated with a deleterious side-effect (e.g., liver, kidney, or blood damage). In such situations, one or more markers for the side-effect can be monitored, and the treatment regimen altered or halted if needed for the safety of the patient.

In view of the uses discussed above, in a further aspect, the invention provides kits comprising the detection reagent of the invention. In general, the kits comprise the detection reagent in at least one container, in combination with packaging materials for storage and shipment of the container. Although the detection reagent may be present in any amounts in the kits, typically the detection reagent is present in an amount that is adequate to practice a method according to the invention at least one time. In embodiments, multiple containers containing detection reagent are supplied in a kit. The detection reagent may be supplied as a pure substance or as part of a composition. Additional components may be supplied in the kit, including, but not limited to, other reagents and supplies for practicing a method of the invention. Those of skill in the art are aware of the various supplies and substances typically included in kits for detecting substances of interest, and any of those supplies and substances may be included in a kit according to the present invention. For example, a kit may comprise one or more solid substrates for performing a method of the invention, which can, in embodiments, be pre-bound by an anchoring molecule (e.g., an antibody) that can specifically bind to the substance of interest. The kit may also or alternatively contain one or more containers (e.g., bottles) holding binding solutions, wash solutions, or covalent cleavage reaction solutions.

In some embodiments, the kits contain some or all of the materials for both binding and detecting a substance of interest. For example, in embodiments, the kit comprises a solid support for binding of a substance of interest (e.g., a microtiter plate, some or all of the wells of which being bound by an antibody), a detection reagent for binding the substance of interest (e.g., a bifunctional complex comprising an antibody and a DNA polymerase), and optionally a reagent for cleavage of the detection reagent. The kit may optionally further comprise a detection reaction vessel for a catalytic reaction (e.g., a PCR reaction tube).

### EXAMPLES

The invention will be further explained by the following Examples, which are intended to be purely exemplary of the invention, and should not be considered as limiting the invention in any way.

### Example 1

### Construction of Streptavidin-Protease Site-Polymerase Fusion Proteins

This Example relates to construction of a subunit of a detection reagent according to the present invention, which comprises both a detection portion and a linker portion. More specifically, this Example pertains to construction of (tethering moiety)-(cleavable site)-(enzyme) proteins for use in construction of a detection reagent of the invention that comprises an antibody. The tethering moiety can be selected from moieties that can bind antibodies either directly, such as protein A, protein G, or via a moiety that can be attached to an antibody, for example streptavidin, which can be tethered via biotin to a biotinylated antibody. The enzyme moiety can be any enzyme that is convenient to detect via its specific activity, such as horseradish peroxidase, alkaline phosphatase, or a polymerase.

In particular, this Example describes construction of (streptavidin)-(protease site)-(polymerase) fusion proteins. Schematic representations of different moieties combined in fusion proteins are depicted in FIG. 1, and with respect to this Example, FIG. 1C in particular. In such a fusion protein, the streptavidin moiety is used to tether the protein to biotinylated antibodies, the protease cleavage site is used to release the polymerase from streptavidin-antibody complex after binding to analyte, and the polymerase moiety is a moiety that is detected or used for signal generation.

Detection of the polymerase moiety is achieved via its ability to produce a copy of a modified polynucleotide template. For example, the template can contain one or more modifications that prevent its amplification in PCR under certain conditions, whereas the copy of the template, which does not contain the modifications, can be amplified. For example, polymerase moiety could be the Klenow fragment of E. coli DNA polymerase I that can copy a modified template containing 2′OMe nucleotides or desoxyuridines or both. The copied template can be amplified in PCR by Pfu polymerase that can not amplify the modified template, but can amplify its copy that has no 2′OMe nucleotides or desoxyuridines. Therefore, the PCR signal resulting from the amplification is proportional to the amount of Klenow moiety bound to the analyte and thus the amount of the analyte.

A DNA fragment encoding a mature streptavidin is obtained in PCR using Streptomyces avidinii DNA as a template and a pair of primers SF1 and SR1:

(SEQ ID NO: 1)

| | |
|---|---|
| SF1 | 5′- GATCCGACCCCTCCAAGGACT -3′ |

(SEQ ID NO: 2)

| | |
|---|---|
| SR1 | 5′- GCGCAGATCTCGAGCTGCTGAACGGCGTCGA -3′ |

The obtained PCR fragment has unique BsaI site shortly downstream of the SF1 primer sequence and unique XhoI and BglII sites encoded in the SR1 primer. The PCR fragment produced using these primers is cleaved at the BsaI restriction endonuclease site situated at the beginning of the nucleotide sequence encoding mature streptavidin. A double-stranded linker having the following double-stranded structure

(SEQ ID NO: 3)

5′- CATGGGCAGCAGCCATCATCATCATCATCAC -3′

(SEQ ID NO: 4)

3′- CCGTCGTCGGTAGTAGTAGTAGTAGTGCGGC -5′

is ligated to that site.

The linker encodes a polyhistidine tag and contains an NcoI sticky end on the one end and a sticky end 5′-CGGC on the another end. The latter is compatible with the sticky end 5′-GCCG that was generated after cleaving the PCR fragment with BsaI. The linker is phosphorylated at its 5′ protruding ends using T4 polynucleotide kinase (New England Biolabs, Beverly, Mass.), and connected to the PCR fragment at its BsaI-generated sticky end using T4 DNA ligase (New England Biolabs, Beverly, Mass.). About 10-100 times excess of the linker over the PCR fragment is recommended to ensure that majority of the PCR fragment has the linker attached. The ligation products are cleaved with NcoI and BglII restriction endonucleases, the linker-PCR fragment is separated from the linker in a 1.5% agarose gel, and the linker-PCR fragment ligation product is purified from the agarose gel, for example using the QIAEX II kit (QIAGEN Sciences, Germantown, Md.) according to the manufacturer's instructions.

The purified DNA fragment is ligated into a pET-15b vector cleaved with NcoI and BamI restriction endonucleases, the ligation products are transformed into XL10 Gold competent cells (Stratagene, La Jolla, Calif.) and seeded on agar plates containing 100 ug/ml ampicillin. The ampicillin-resistant colonies emerge after incubating the plates at 37° C. overnight, the plasmid DNA is extracted by boiling a material from a colony in 1 ml of water for 3 minutes, and the released plasmids are analyzed for the presence of the insert in PCR using SF1 and SR1 primers. The insert-positive colonies are grown overnight in liquid medium, such as L-broth supplemented with 100 ug/ml ampicillin, and the plasmid DNAs are purified using StrataPrep® Plasmid miniprep kit (Stratagene, La Jolla, Calif.). The DNA sequence of the obtained plasmids can be verified by Sanger method using a T7 promoter primer:

| | |
|---|---|
| 5′- TAATACGACTCACTATAGG -3′ | (SEQ ID NO: 5) |

that is complementary to the T7 promoter DNA sequence upstream of the insert.

The resulting pET-SAV plasmid encodes His-tagged straptavidin under control of T7 promoter. A unique XhoI site encoded in the SR1 primer is introduced at the end of streptavidin ORF in order to facilitate cloning of DNA sequences encoding a protease cleavage site and a polymerase in lieu of the streptavidin gene.

Unstructured flexible regions that are adjacent to the protease sites are known to facilitate cleavage at the sites because they often make the sites more accessible to proteases. In the molecule depicted in FIG. 1C, a synthetic flexible region that is rich with small amino acids, for example glycine, serine, alanine, or threonine, is present adjacent to the protease cleavage site.

A double-stranded oligonucleotide, referred to herein as GS, that encodes small amino acids glycine and serine, having the following double-stranded structure, is synthesized:

(SEQ ID NO: 6)

5′- TCGACGGATCGGGCGGTGGCTCCGGTGGCGGCAGCGGCC -3′

(SEQ ID NO: 7)

3′- GCCTAGCCCGCCACCGAGGCCACCGCCGTCGCCGGAGCT -5′

The pET-SAV plasmid is cleaved with XhoI endonuclease at the end of the nucleotide sequence encoding SAV ORF and the GS oligonucleotide that is flanked by XhoI sticky ends (5′-TCGA) is ligated into that site. The resulting clones are sequenced using the T7 promoter primer, and the clones that have the GS oligonucleotide in correct orientation in-fusion with SAV ORF are selected for further work. A selected clone was designated pET-SAV-GS. This clone has unique XhoI site at the end of the GS sequence, as the XhoI sticky end at the beginning of the GS does not result in complete XhoI site when ligated into the plasmid. The unique XhoI site is used to add further moieties downstream of SAV-GS fusion gene. This approach, i.e., inserting polynucleotide sequences that result in unique XhoI site at the end of the inserted sequence, is used repeatedly in this Example to add more and more polynucleotide sequences encoding moieties of interest.

In an embodiment not depicted in FIG. 1C, a naturally occurring flexible unstructured region is used instead of the GS oligonucleotide or in addition to the GS oligonucleotide. Such regions can be selected from abundantly expressed proteins, for example from L7/L12 ribosomal protein (Bocharov E V, et al., J. Biol. Chem. 279(17):17697-706, 2004). Many unstructured regions are known to those skilled in the art and are available from protein structures in databases, for example from Entrez Structure NCBI database. Furthermore, unstructured regions can be predicted in proteins by bioinformatics methods, for example using a Scooby-Domain method (Pang, C N, et al., Nucleic Acids Res. 36(2):578-88, 2008).

A PCR fragment encoding flexible unstructured region of the protein is obtained using E. coli K12 DNA as a template and L73F and L71R pair of primers:

(SEQ ID NO: 8)

| | |
|---|---|
| L73F | 5′- GCGCGTCGACGAAGAAAAATTCGGTGTTTCC -3′ |

(SEQ ID NO: 9)

| | |
|---|---|
| L71R | 5′- GCGCCTCGAGCTTTTCTTCAGCAGCTTCAACC -3′ |

The L73F primer encodes a SalI restriction site and the L71R primer encodes a XhoI restriction endonuclease site. The resulting L7 PCR fragment has a SalI site at the beginning and a XhoI site and the end of partial L7/L12 ORF that is encoded in the fragment. The fragment is cleaved with XhoI and SalI restriction endonucleases and ligated into a pET-SAV-GS plasmid cleaved with XhoI. The plasmids with the correct orientation of the insert are selected using DNA sequencing as above. The resulting plasmid was designated pET-SAV-GS-L7. Several tandem copies of the L7 fragment can be inserted in lieu of streptavidin moiety using this approach. The resulting plasmids with one or more L7 inserts has a unique XhoI site at the end of the L7 ORF. As discussed above, a unique XhoI site is used to add more moieties downstream of the fusion gene.

Next, a double stranded oligonucleotide that encodes a protease cleavage site is introduced into the unique XhoI site of the pET-SAV-GS-L7 or pET-SAV-GS plasmid DNA. Non-limiting examples of suitable protease cleavage sites are given in Table 1. The design of the double-stranded oligonucleotides encoding the cleavage sites is similar; they have XhoI-compatible sticky ends on both sides, but only the downstream end provides for creation of XhoI site when the oligonucleotides are ligated into the XhoI cut plasmid.

TABLE 1
Exemplary Proteases and Cleavage Sites

| | Cleavage |
|---|---|
| Excision site ↓ | Enzyme/Self-Cleavage |
| Asp-Asp-Asp-Asp-Lys↓ (SEQ ID NO: 28) | Enterokinase |
| Ile-Glu/Asp-Gly-Arg↓ (SEQ ID NO: 29) | Factor Xa protease |
| Leu-Val-Pro-Arg↓Gly-Ser (SEQ ID NO: 30) | Thrombin |
| Glu-Asn-Leu-Tyr-Phe-Gln↓Gly (SEQ ID NO: 31) | TEV protease |
| Leu-Glu-Val-Leu-Phe-Gln↓Gly-Pro | PreScission ™ |
| (SEQ ID NO: 32) | protease |
| Specific intein-encoded sequences | Intein 1 & intein 2 |

In particular, sites for proteases that have high cleavage specificity are preferred, for example, tobacco etch virus (TEV) protease or Prescission™ protease, in order to minimize cleavage in the enzyme moiety of the fusion protein and reduction of signal. However, less specific proteases, such as Enterokinase, Factor Xa protease, and Thrombin can be used, provided they do not significantly affect integrity of the enzyme moiety, such as a polymerase. Self-cleavable proteases, such as intein-encoded proteases, could be used also.

An additional molecule for use in this Example can be created. Specifically, a double-stranded oligonucleotide referred to herein as TS, encoding a naturally occurring TEV polyprotein TEV protease cleavage site is synthesized from the following double-stranded nucleic acid structure:

(SEQ ID NO: 10)

5′- TCGAAGGCACCACAGAGAACCTGTACTTTCAGAGCGGCACCC -3′

(SEQ ID NO: 11)

3′- TCCGTGGTGTCTCTTGGACATGAAAGTCTCGCCGTGGGAGCT -5′

Yet another molecule for use in this Example can be produced. Specifically, a double-stranded oligonucleotide, referred to herein as PS, encoding a Prescission™ protease cleavage site is synthesized using the following double-stranded nucleic acid structure:

(SEQ ID NO: 12)

5′- TCGATGTGCTGTTTCAAGGTCCAGGATCCGGTC -3′

(SEQ ID NO: 13)

3′- ACACGACAAAGTTCCAGGTCCTAGGCCAGAGCT -5′

pET-SAV-GS plasmid is cleaved with XhoI endonuclease at the end of the nucleotide sequence and the TS or PS oligonucleotide is ligated into that site. The resulting clones are sequenced using the T7 promoter primer, and the clones that have the TS or PS oligonucleotide in correct orientation in-fusion with SAV-GS ORF are selected for further work. Selected clones were designated pET-SAV-GS-TS and pET-SAV-GS-PS. Clone pET-SAV-GS-TS has a unique XhoI site after the nucleotide sequence encoding TEV protease cleavage site, and clone pET-SAV-GS-PS has a unique XhoI site after the nucleotide sequence encoding Prescission™ protease cleavage site. The XhoI sites are used to add further moieties downstream of SAV-GS-TS and SAV-GS-PS fusion genes.

Similarly, pET-SAV-GS-L7 plasmid is cleaved with XhoI endonuclease at the end of the nucleotide sequence and the TS or PS oligonucleotide ligated into that site. The resulting clones are sequenced using the T7 promoter primer, and the clones that have the TS or PS oligonucleotide in correct orientation in-fusion with SAV-GS-L7 ORF are selected for further work. Selected clones were designated pET-SAV-GS-L7-TS and pET-SAV-GS-L7-PS. Clone pET-SAV-GS-L7-TS has a unique XhoI site after the nucleotide sequence encoding the TEV protease cleavage site, and clone pET-SAV-GS-L7-PS has a unique XhoI site after the nucleotide sequence encoding the Prescission™ protease cleavage site. The XhoI sites are used to add further moieties downstream of SAV-GS-L7-TS and SAV-GS-L7-PS fusion genes.

For creation of one molecule in this Example, a flexible unstructured amino acid sequence can be added after the protease cleavage site to provide large unstructured regions on both sides of the cleavage site in order to facilitate the cleavage. For example, the above-described L7 PCR fragment can be cleaved with SalI and XhoI restriction and ligated into either pET-SAV-GS-PS or pET-SAV-GS-TS plasmids cleaved with XhoI. The resulting plasmids have been designated pET-SAV-GS-PS-L7 and pET-SAV-GS-TS-L7. They have unique XhoI sites at the end of SAV-GS-PS-L7 and SAV-GS-TS-L7 fusion genes that are used to clone an enzyme moiety. As mentioned above, the enzyme moiety can be a polymerase, a horseradish peroxidase, alkaline phosphatase, or essentially any enzyme that is useful in immunodetection methods. For example, the Klenow fragment of DNA polymerase I can be cloned at the end of the fusion genes.

A PCR fragment encoding Klenow is generated using E. coli DNA as a template and a pair of primers:

(SEQ ID NO: 14)

KlinF
5′- GCGCGTCGACGGTGGCGGTGGCTCGGTGATTTCTTATGACAACTAC
GTCA - 3′

(SEQ ID NO: 15)

KWR
5′- GCGCAAGCTTAGTGCGCCTGATCCCAGTTTTC -3′

The resulting PCR fragment is designated KL. It encodes the entire Klenow ORF preceded by several codons encoding glycines and serines that were designed in the KlinF primer to provide additional distance between Klenow and protease site moieties and a better accessibility for a protease. The fragment is cut at both ends at SalI and HindIII sites provided in the primers and cloned into any of the plasmids, pET-SAV-GS-TS, pET-SAV-GS-PS, pET-SAV-GS-L7-TS, pET-SAV-GS-L7-PS, pET-SAV-GS-PS-L7, pET-SAV-GS-TS-L7 that were cleaved with XhoI and HindIII. Several small amino acids are encoded in the KlinF primer to add flexibility in between protease sites and the enzyme. The resulting plasmids were designated SAV-GS-TS-KL, SAV-GS-PS-KL, pET-SAV-GS-L7-TS-KL, pET-SAV-GS-L7-PS-KL, pET-SAV-GS-PS-L7-KL, pET-SAV-GS-TS-L7-KL.

In yet another procedure for creating molecules for the invention, an affinity purification tag is added at the end of the fusion genes. This C-terminal tag provides for tandem affinity purification (TAP) in combination with the His-tag that was introduced at the N-terminus of streptavidin. There are many tags are known to those skilled in the art that can be used for TAP, that are either based on binding to its natural or modified small ligand or a protein binding partner, or on binding to immobilized tag-specific antibodies. By the way of example, FLAG, His, CBP, CYD (covalent yet dissociable NorpD peptide), Strep II, HPC (heavy chain of protein C) peptide tags, and the GST and MBP can be combined for TAP.

To provide a FLAG tag on the C-terminus of the Klenow (KLF), a double-stranded oligonucleotide F encoding two FLAG tags followed by a stop codon is synthesized using the following double-stranded nucleic acid structure (SEQ ID NO:16 top line; SEQ ID NO:17 bottom line):

5′- CTAGTGATTATAAGGATGACGATGACAAAGATTACAAAGATGATGA
CGATAAGTAG -3′
3′- ACTAATATTCCTACTGCTACTGTTTCTAATGTTTCTACTACTGCTA
TTCATCTTAA -5′

Oligonucleotides encoding less than two or more than two FLAG tags can be used also. In order to remove a stop codon at the end of Klenow ORF, Klenow DNA template is amplified with a pair of primers:

(SEQ ID NO: 18)

| | |
|---|---|
| XhoF | 5′- CGTGCACTCGAGTTGCTAAA -3′ |

(SEQ ID NO: 19)

| | |
|---|---|
| SpR | 5′- GCGCACTAGTATGCGCCTGATCCCAGTTTTC - 3′ |

The obtained PCR fragment has unique internal XhoI site that resides in the Klenow polynucleotide sequence and unique SpeI site that was introduced at the end of the Klenow ORF before the stop codon using the SpR primer. The fragment is cleaved with XhoI and ligated onto any of the pET-SAV-GS-TS-KL, pET-SAV-GS-PS-KL, pET-SAV-GS-L7-TS-KL, pET-SAV-GS-L7-PS-KL, pET-SAV-GS-PS-L7-KL, pET-SAV-GS-TS-L7-KL plasmids cleaved with XhoI at the same internal site. Next, T4 DNA ligase is inactivated by incubation at 65° C. for 20 min, the ligation products are cleaved with SpeI and EcoRI restriction endonucleases according to the manufacturer's instructions, separated in 1% agarose gel, and the largest DNA fragment, containing entire pET-15b DNA sequence between NcoI and EcoRI sites, and a fusion gene connected at the NcoI site to the plasmid, including Klenow gene up to the SpeI site at the end of it is purified, for example using QIAEX II kit (QIAGEN Sciences, Germantown, Md.) according to the manufacturer's instructions. Oligonucleotide F that is flanked by SpeI and EcoRI sticky ends is ligated with the purified fragment, that has the same sticky ends, the ligation products are transformed into XL10 Gold competent cells (Stratagene, La Jolla, Calif.) and seeded on agar plates containing 100 ug/ml ampicillin. The ampicillin-resistant colonies emerge after incubating the plates at 37° C. overnight, the plasmid DNA is extracted by boiling a material from a colony in 1 ml of water for 3 minutes, and the released plasmids are analyzed for the presence of the insert in PCR using XhoF and R (5′-CTACTTGTCATCGTCATCCTTAT-3′ (SEQ ID NO:20)) primers. The insert-positive colonies are grown overnight on liquid medium, such as L-broth supplemented with 100 ug/ml ampicillin, and the plasmid DNAs are purified using StrataPrep® Plasmid miniprep kit (Stratagene, La Jolla, Calif.). The presence of double FLAG tag at the end of the fusion genes is verified by Sanger method using MfeF (5′-CGGCAGAAGTGTTTGGTTTG-3′ (SEQ ID NO:21)) primer.

The resulting plasmids were designated SAV-GS-TS-KLF, SAV-GS-PS-KLF, pET-SAV-GS-L7-TS-KLF, pET-SAV-GS-L7-PS-KLF, pET-SAV-GS-PS-L7-KLF, pET-SAV-GS-TS-L7-KLF.

### Example 2

### Construction of Streptavidin-Protease Site-Phosphatase Fusion Proteins

Traditionally, calf intestine alkaline phosphatase is widely used as a detectable moiety for immunodetection of analytes, for example in ELISA. However, a wide variety of alkaline phosphatases (AP) can be used for immunodetection, for example mammalian (bovine, human), invertebrate (shrimp), or of microbial origin. Alkaline phosphatases are present in most, if not all organisms. Alkaline phosphatases with high specific activity are preferred, for example Cobetia marina alkaline phosphatase.

By way of example, a polynucleotide sequence encoding Geobacillus thermodenitrificans strain T2 alkaline phosphatase (NCBI accession EU239359) is amplified in PCR using Geobacillus thermodenitrificans DNA as a template and T2F and T2R primers:

(SEQ ID NO: 22)

| | |
|---|---|
| T2F | 5′- GCGCCTCGAGCATGGCGGACAAGCAGTAT -3′ |

(SEQ ID NO: 23)

| | |
|---|---|
| T2R | 5′- GCGCAAGCTTCTATGGCCTCACGACACATT -3′ |

The PCR product is digested at both ends with XhoI and HindIII restriction endonucleases. Alternatively, oligonucleotides encoding ORF resident in the fragment can be synthesized, annealed and amplified in PCR using the same primers.

pET-SAV-GS-PS, pET-SAV-GS-TS, pET-SAV-GS-L7-PS, pET-SAV-GS-L7-TS plasmids that were described in Example 1 are also cleaved with Xho I and Hind III restriction endonucleases and the PCR product is ligated with these plasmids. The resulting plasmids pET-SAV-GS-PS-T2, pET-SAV-GS-TS-T2, pET-SAV-GS-L7-PS-T2, pET-SAV-GS-L7-TS-T2 encode Geobacillus thermodenitrificans alkaline phosphatase in fusion with streptavidin moiety and separated from the moiety by a flexible unstructured region harboring a protease cleavage site.

### Example 3

### Production of Fusion Proteins

E. coli strains harboring any of the plasmids encoding SAV fusion proteins described in Examples 1 and 2 are used for production of the streptavidin fusion proteins described in the Examples. Routinely, E. coli BL21 DE3 strain (Stratagene, La Jolla, Calif.) is used; however, any E. coli strains that express T7 RNA polymerase are suitable for this purpose because the expression of the fusion proteins is driven by a T7 promoter residing in the pET-15b vector. However, other vectors and other promoters, for example tac promoter, can be used to produce the proteins. Cultivation of recombinant E. coli for expression of proteins under control of T7 promoter is performed essentially as disclosed in U.S. Pat. No. 4,952,496. The cells are harvested by centrifugation at 5,000 rpm for 15 min at 4° C. in a JA-10 rotor using a J2-21 centrifuge (Beckman Coulter, Fullerton, Calif.). The cells from 250 ml culture are resuspended in 10 ml of buffer A (50 mM Tris-HCl, pH 8, 150 mM NaCl, 5 mM dithiothreitol) supplemented with 1% triton X-100 and a set of protease inhibitors (“Complete”; Roche Diagnostics GmbH, Mannheim, Germany), and disrupted by constant sonication for 1.5 min using a Sonifier 250 at output 3 setting (Branson Ultrasonics Corporation, Danbury, Conn.).

The disrupted cells are centrifuged at 20,000 rpm for 15 min at 4° C. in the JA-20 rotor using the J2-21 centrifuge (Beckman Coulter, Fullerton, Calif.). Streptavidin fusion proteins form inclusion bodies and are found in the pellet. However, significant amounts of soluble proteins could be recovered, especially if streptavidin and an enzyme are separated by long flexible unstructured regions. Nevertheless, purification from the insoluble fraction is preferred since the pellet is enriched in the fusion proteins as compared to supernatants, the fusion proteins in the pellet are less prone to protein degradation, and most importantly, they need to be denatured anyway to remove endogenous biotin. The pellet is resuspended in 10 ml of buffer A by sonication as described above, diluted in buffer A to 40 ml and centrifuged again at 20,000 rpm for 15 min at 4° C. in the JA-20 rotor in the J2-21 centrifuge.

The pellet is resuspended in 7.5 ml of buffer A and the purification is performed from 300 ul of the resuspended inclusion bodies. The rest of the pellet is stored at −80° C. for future purifications. A 300 ul aliquot of the resuspended inclusion bodies is centrifuge for 15 min at 14,000 rpm/min in a microfuge, supernatant is discarded and the pellet is resuspended in 2.8 ml of 6M GdHCl pH 1.5, and transferred into two 1.5 ml tubes. The tubes are rotated at +4° C. for 1 h after which they are centrifuged in a microfuge for 15 min at 14,000 rpm/min.

The supernatant is collected and dialyzed against 300 ml of 6M GdHCl pH 1.5 for overnight at room temperature in order to remove biotin that was bound to the streptavidin moiety. Further dialyzis steps are performed at +4° C.

Next, the sample is dialyzed against 300 ml of 6 M GdHCl, 0.5 M NaCl, 20 mM Na—P pH7.4 for 4 h, and then further dialyzed against three changes of the same volume of buffer B (20 mM sodium phosphate pH 8, 150 mM NaCl, 2 mM dithiothreitol) for at least 2 h each change.

The sample is removed from the dialysis bag, centrifuged in a microfuge as before and the supernatant is collected into 1.5 ml microtubes. Imidazole is added to the supernatant to 10 mM concentration, after which 100 ul of the 50% slurry of Ni-NTA agarose beads (QIAGEN Sciences, Germantown, Md.) equilibrated in buffer B is added to each 1.4 ml of the supernatant. The supernatant is rotated with the beads at +4° C. for 15 min, after which the beads are pelleted by centrifugation at 2,000 rpm/min for 2 min, washed three times with 1.5 ml of the buffer B supplemented with 10 mM imidazole, pelleted as described above and the supernatant completely removed. The adsorbed fusion proteins are eluted with 250 mM imidazole in buffer B and dialyzed overnight against at least 100 volumes of buffer containing 30 mM sodium phosphate pH 7.2, 50 mM NaCl, 4 mm DTT, 50% glycerol. The purified protein is stored at −20° C.

### Example 4

### Detection of Analyte Using Streptavidin-Protease Site-Klenow Proteins

Commercially available kits comprising capture antibodies, analytes, and biotinylated detection antibodies are used to test performance of streptavidin-protease site-klenow fusion proteins. Human EGF (cat# DY236) and human VEGF (cat# DY293B) kits from R&D Systems, Minneapolis, Minn., are used according to the manufacturer's instructions for overnight coating of capture antibody, blocking, analyte dilution and capture, washing, and application of biotinylated detection antibody. After addition of the detection antibody, a streptavidin-protease site-klenow protein described in Example 1 is added instead of the SAV-HRP from the commercial kit.

First, the fusion protein is diluted to 100 ng/ml in Blocking/dilution buffer containing 0.2% fish skin gelatin, 2% heat inactivated normal goat serum, 10 mM Tris-HCl pH 8.0, 200 mM NaCl, 0.02% Tween-20. The diluted protein is added to the ELISA plate at 75 ul/well and incubated for 30 min at room temperature to allow the SAV moiety to bind to the biotinylated antibody. Then the solution is aspirated from the wells and wells are washed 4 times with 400 ul of TBST buffer (25 mM Tris-HCl pH 8.0, 125 mM NaCl, 0.1% Tween-20), followed by 2 washes with deionized water. At the next step, the polymerase moiety is cleaved from the fusion protein. The PreScission™ protease is used in case the fusion protein contains a cleavage site for the protease. The water is aspirated from the wells and 100 ul of PreScission™ potease cleavage solution is added to the wells. The solution contains 50 mM Tris-HCl, pH 7.0 at 25° C., 100 mM NaCl, 0.5 mM EDTA, 0.2 mM DTT, and 0.05 units per ul of PreScission™ protease (GE Healthcare, Piscataway, N.J.). The reaction is allowed to continue for 1-16 h at constant shaking at 5° C., or for 0.5-3 h at room temperature with constant shaking.

Alternatively, TEV protease is used in case where the fusion protein contains a TEV cleavage site. The water is aspirated from the wells and 100 ul of TEV protease cleavage solution is added to the wells. The solution contains 50 mM Tris-HCl, pH 8.0, 0.5 mM EDTA, 0.2 mM DTT, and 20 units of TEV protease (Invitrogen, Carlsbad, Calif.) per well. The reaction is allowed to proceed for 0.5-2 h at rt at constant shaking. Alternatively, the reaction is performed at 30° C. for the same period of time.

After completing the TEV or PreScission™ protease reactions, 2 ul of a reaction mixture that contains Klenow polymerase cleaved from the streptavidin moiety is transferred into a PCR plate (ABgene, Epsom, United Kingdom) wells with 11 ul of Klenow extension mix:

Klenow Extension Mix (all Conc. are Final after Mixing)

10 mM Tris-HCl (pH 7.5)

5 mM MgCl2

7.5 mM DTT

100 ug/ml BSA

500 uM dNTP (ACGT)

100 nM Alien1-Reverse primer

40 pM Oligo1

(SEQ ID NO: 24)

Oligo1:
5′- TTTTTTTGCTCGACGGTGAAUGAUGTAGGUACCAGC
AGUAACUCGAGCACGUCUU
2′OMe (CG)A2′OMe (CC)AAATCUGGAUATTGCAGCCTCGT -3′

(where 2′OMe indicates 2′-OMe nucleotides; U indicates 2′-deoxyuridine).

Alien1-Rev primer (anneals to 3′ end of Oligo1 to prime Klenow reaction):

| | |
|---|---|
| 5′- ACGAGGCTGCAATATCCAGA -3′ | (SEQ ID NO: 25) |

The reaction is allowed to proceed for 0.5-16 h at room temperature. During the reaction, Klenow polymerase produces a copy of the Oligo1 template that has no 2′-Me or 2′-deoxyuridine modifications.

Next, a 2×QPCR MM mix is added to the wells to provide for the following concentrations of the components of PCR reaction:

15 mM Tris-HCl pH 8.4

50 mM KCl

2.5 mM MgCl₂

3% DMSO

0.01% Tween-20

800 nM dNTPs (ACGT)

0.444×SYBR Green (Molecular Probes, provided as 10,000×)

30 nM Rox reference dye (Stratagene Cat#600530)

Pfu(exo-) 50 U/ml (Stratagene Cat#600163-81)

| | |
|---|---|
| 100 nM Alien1-Forward primer | |
| (5′- TGCTCGACGGTGAATGATGT -3′ | (SEQ ID NO: 26) |
| 100 nM Alien1-Reverse primer | |
| (5′- ACGAGGCTGCAATATCCAGA -3′ | (SEQ ID NO: 27) |

After mixing the samples, they are run in an MX3005P using the program for SYBR green with dissociation curve and 2-step cycling parameters of 95° C. for 10 min (1 cycle), 95° C. for 15 sec then 63° C. for 45 sec (40 cycles). The relative QPCR signal is computed by calculating the change in Ct between the Ct at analyte concentration=0 pg/ml and the Ct at analyte concentration=X pg/ml. This is called the “dCt from 0”. In a 100% efficient assay, a change of one Ct is equivalent to a doubling of the amount of initial usable QPCR template. To convert this logarithmic signal into a linear quantity the following formula “Relative QPCR Signal”=2̂ (dCt from 0)−1 is used. Finally, relative QPCR Signal is plotted against the concentration of analyte (pg/ml) used in a sample.

### Example 5

### Detection of Analyte Using Releasable DNA Conjugated to Binding Moieties

This example pertains to use of (tethering moiety)-(cleavable DNA bridge)-(nucleic acid) protein-DNA conjugates. Tethering moieties can be selected from moieties that can bind antibodies either directly, such as protein A, protein G, or via a moiety that can be attached to an antibody, for example streptavidin which can be tethered via biotin to a biotinylated antibody. Detectable nucleic acid can be any sequence that is convenient to detect via PCR or other means.

In particular, this example describes the use of streptavidin-DNA constructs containing restriction endonuclease site useful in releasing a DNA strand from the antibody. The DNA strand may be linked on its other end to a catalytic moiety for use in a detection reaction. In the conjugate, a streptavidin moiety is used to tether the protein to biotinylated antibodies, the DNA bridge contains a restriction endonuclease site that is used to release at least a portion of the DNA strand from streptavidin-antibody complex after binding to analyte. After cleavage, the catalyst attached to a portion of the DNA bridge is removed to a new container (i.e., a detection vessel) and used in a reaction that generates a detectable signal.

In this Example, the catalyst is a polymerase and detection is achieved via PCR. The original SAv-conjugated nucleic acid may be double stranded or may be single stranded. If it is single stranded it may be rendered double stranded by the action of a DNA polymerase or by binding of a complementary strand, which itself is bound to the polymerase. For example, the polymerase used for PCR could be Pfu polymerase. In this case, the PCR signal is proportional to the amount of polymerase released, which is proportional to the amount bound to the analyte and thus to the amount of the analyte itself.

Referring now generally to FIG. 2, an analyte is used in an ELISA format (direct or sandwich). The analyte and is bound by a pre-formed detection reagent of the invention or by an Ab that is/becomes linked to a DNA tether or linker, which itself becomes/is linked to a polymerase to provide a detection reagent according to the invention. Excess reagent is washed away. The DNA linker is then covalently cleaved by a dsDNA restriction endonuclease at a pre-defined cleavage site, and the polymerase (with remaining portion of DNA linker) goes into solution. The polymerase is then moved into a PCR reaction tube where it makes a complementary strand from a DNA template which is then amplified. Monitoring by QPCR establishes how much polymerase was transferred to the PCR reaction. In this system, a detectable signal from PCR is generated substantially from polymerase that 1) is part of a detection reagent that specifically bound to the analyte via the antibody portion, and 2) that is cleaved from the detection reagent to release a soluble polymerase portion. In this way, polymerase that non-specifically sticks to the side of the ELISA plate will not participate in the PCR reaction, providing the assay with greater specificity (i.e., better signal-to-noise ratio).

### Example 6

### Chemical Coupling of Oligonucleotides to Proteins for Tethering or Detection

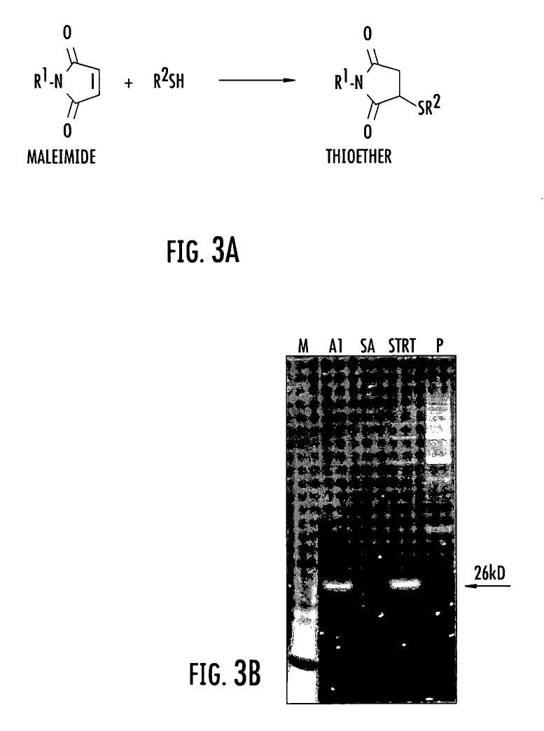

Antibodies, proteins, and other macromolecules can be labeled with heterobifunctional cross-linking agents using commercially available kits (Pierce/Thermo Scientific, Rockford, Ill.). An exemplary heterobifunctional agent, maleimide, is depicted in FIG. 3A, and is referenced in this Example. In general, heterobifunctional crosslinkers possess two or more different reactive groups that allow for sequential conjugations with specific functional groups (e.g., primary amines), minimizing undesirable polymerization or self-conjugation. In this Example, streptavidin was labeled with a heterobifunctional cross-linking agent generating free maleimide groups. Maleimide reacts specifically with sulfhydryl groups at neutral pH. An oligonucleotide synthesized with a sulfhydryl group was then coupled to the streptavidin via the maleimide. The preparation of the streptavidin-oligonucleotide conjugate can be broken down into three parts: 1) reduction of the sulfhydryl group on the oligonucleotide; 2) coupling of the reduced oligonucleotide to streptavidin maleimide; and 3) affinity purification of the conjugate over 2-iminobiotin sepharose.

An 80mer oligonucleotide with a 5′ thiol modification was purchased from Integrated DNA Technologies (Coralville, Iowa). Streptavidin-maleimide was purchased from Sigma (St. Louis, Mo.) and resuspended in PBS, 5 mM EDTA to a concentration of 1 mg/ml. Dithiothreitol (DTT), Triethylamine (TEA), 2-mercaptoethanol, sodium carbonate, sodium acetate, and fish gelatin from cold water fish skin, were also purchased from Sigma. Micro BioSpin 6 desalting columns and Poly-Prep chromatography columns were purchased from BioRad (Richmond, Calif.). VivaSpin 500 concentrators were purchased from VivaScience (Sartorius Group, Hannover, Germany), and Slide-A-Lyzer Mini Dialysis Units (10,000 mwco) were purchased from Pierce (Rockford, Ill.). 2-iminobiotin sepharose was prepared by Derek Hall at BioCrest (Basstrop, Tex.).

Prior to setting up the reduction reactions, six Micro BioSpin 6 columns were washed four times with 0.5 ml PBS as follows. The caps were removed from both ends of the columns and the solution was allowed to drain into a microfuge tube by gravity flow. The solution was discarded and the columns were placed back into their carrier tubes. The columns were then centrifuged for 4 minutes at 4000 rpm in a microfuge. The eluate was discarded from the carrier tube and the column was replaced. 0.5 ml of PBS was applied to each column bed and the spin was repeated. This PBS wash was performed a total of four times. The columns were set aside in clean carrier tubes until needed.

Prior to setting up the reduction reactions, the oligonucleotide was resuspend in HPLC grade water to a final concentration of 100 uM, and fresh IM DTT was prepared. Three of the following reactions were performed per SA-A1 preparation in a total of 50 ul:

| | | |
|---|---|---|
| 10 | ul | 100 uM 5′thiol-oligo (1 nMole) |
| 36.5 | ul | HPLC grade water |
| 1 | ul | TEA |
| 2.5 | ul | 1M DTT |

The reactions were allowed to incubate at room temperature for 30 minutes. During this incubation, three 15 ug aliquots of streptavidin-maleimide were removed from −80° C. and thawed at room temperature. The DTT was removed from the reduction reactions by two serial purifications on the pre-washed desalting columns. Each 50 ul reduction reaction was loaded onto one column and centrifuged for 4 minutes at 400 rpm in a microfuge. Eluates were then loaded onto the remaining clean columns and the spin was repeated. A 15 ug aliquot of streptavidin-maleimide was immediately added to the eluate in the spin column catch tube. The reduced Alien1 oligos can form dimers so it was important to minimize the time between desalting and the addition of the streptavidin. Reactions were mixed by vortexing and transferred to a 37° C. water bath. Coupling reactions proceeded for 2 hours and were then quenched by the addition of 0.5 ul of 0.5M 2-mercaptoethanol. The three reactions were combined into one tube prior to affinity purification.

A 250 ul drip column of 2-iminobiotin was packed in a Poly-Prep chromatography column and was equilibrated at room temperature by washing with 10 ml of binding buffer (50 mM sodium carbonate pH11, 0.5M NaCl). Four volumes of binding buffer were added to the combined pool of the conjugate to bring the pH up to 11. A small volume of this mix was set aside and labeled “start” to be used for later analysis. The remainder of the sample was loaded onto the column and the eluate was collected in a clean microfuge tube. A new tube was placed under the column and the eluate was passed through the column again. This process was repeated two additional times until the sample had been passed through the column a total of four times. The final eluate was collected and labeled “flow thru” and stored at 4° C. The charged column was then washed with 10 ml of binding buffer, the first 2 ml were collected and labeled “wash 1” and “wash 2”, respectively. The SA-A1 was eluted from the column with 2 ml of elution buffer (50 mM sodium acetate pH4, 0.5M NaCl). The 2 ml eluate was then concentrated using VivaSpin 500 spin concentrators (centrifuged at 15,000×g for 5 minutes), 0.5 ml per concentrator. The four concentrates were collected and combined. The volume of the combined concentrates was adjusted up to 500 ul with 10 mM Tris pH7.5 and transferred back to one of the previously used concentrator units and the concentration was repeated. The concentrate was collected and the volume was adjusted up to 50 ul by the addition of 10 mM Tris pH7.5. This sample was then dialyzed for 2 hours at 4° C. against 1 L of 10 mM Tris pH7.5 in a Slide-A-Lyzer mini dialysis cup. Following dialysis, the pH of the sample was confirmed to be 7.5 by using a pH strip. 1% sodium azide was added to a final concentration of 0.02%. The purified conjugate was stored at 4° C.

To assess the extent of conjugation and purity an electrophoretic analysis was performed. The unconjugated oligonucleotide (25 ng), unconjugated streptavidin-maleimide (250 ng), the conjugated but unpurified “start” fraction from the affinity purification (2 ul), and the purified conjugate (2 ul of the concentrated stock) were analyzed on a NuPAGE 4-12% acrylamide gradient denaturing gel (Invitrogen, Carlsbad, Calif.). Samples were prepared in NuPAGE sample buffer supplemented with 1 mM DTT and boiled for 2 minutes prior to being loaded on the gel. Following electrophoretic separation, the gel was soaked in water with gentle rocking twice for 15 minutes. The gel was then stained with SYBR gold (Invitrogen/Molecular Probes) for 30 minutes. The gel was rinsed briefly with water and photographed under ultraviolet illumination and a SYBR stain filter. The results are shown in FIG. 3B.

More specifically, denaturing protein gel analysis was used to confirm that coupling between the streptavidin-maleimide and the oligonucleotide occurred, and that the majority of the free oligonucleotide was eliminated from the final preparation. The gel was stained with SYBR gold to visualize only the nucleic acids. In lane “A1”, uncoupled Alien1 oligonucleotide is clearly visible, and is also present in the coupled but unpurified “Strt” fraction. No uncoupled oligonucleotide band is visible in the purified fraction “P”, indicating that the majority was eliminated by the affinity purification.

Streptavidin exists in nature as a 60 kD tetramer so one would anticipate observing a 15 kD solitary band on a denaturing protein gel. The laddering observed is a result of the maleimide derivatization, which results in a degree of covalent cross-linking of the streptavidin molecules. Once the oligonucleotide (26 kD) is coupled, a general shift upward in molecular weight is anticipated as is the positive staining with SYBR gold.

### Example 7

### Construction of Streptavidin-Cleavable DNA Bridge-Polymerase Conjugates

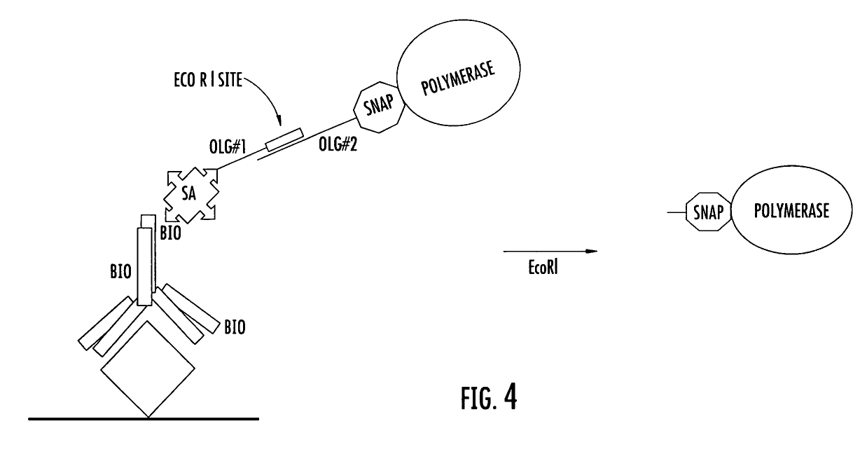

A cleavable DNA bridge can be constructed through the annealing of two complimentary single stranded oligonucleotides each tethered to a separate protein. In this Example, one protein-DNA conjugate is a streptavidin-oligo1 conjugate produced by covalently linking a single-stranded oligonucleotide to streptavidin. This is annealed to a polymerase-oligo2 conjugate in which oligo2 is partially complimentary to the oligo 1. Upon hybridizing, the two oligos form a DNA bridge between the proteins of a detection reagent comprising an antibody binding region and a polymerase detection region. The bridge or linker contains a reconstituted restriction enzyme site, EcoR1. This concept is generally depicted in FIG. 4, which is presented with reference to “SNAP tag” technology, as discussed below.

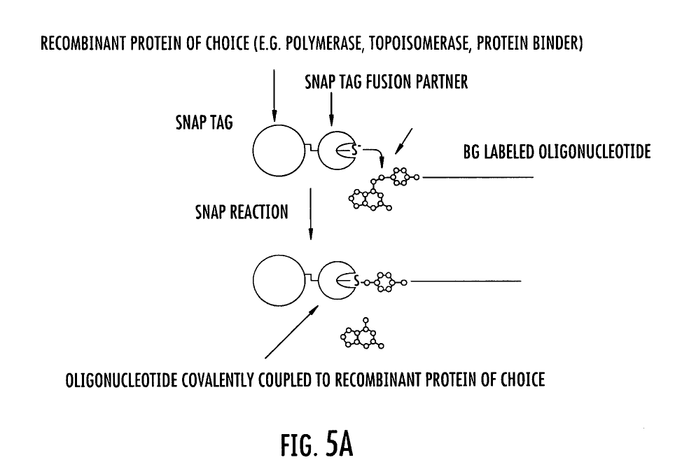

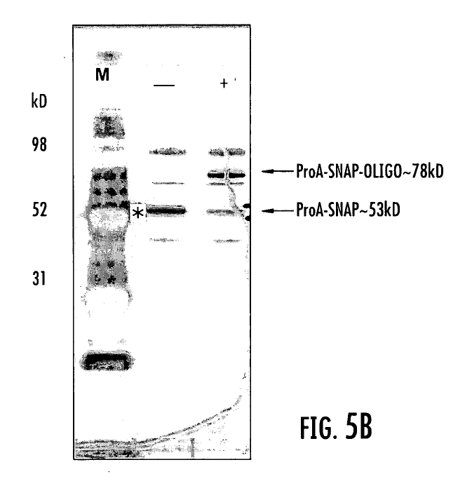

In addition to indirect chemical coupling methods that link molecules to any available reactive site, oligonucleotides (or other molecules) can be covalently coupled to a recombinant protein of interest in a directed fashion using SNAP tag technology (Covalys, Benkenstrasse, Switzerland). The SNAP tag is based on alkylguanine-DNA-alkyltransferase “AGT”, which performs an irreversible reaction with benzylguanines. Any desired coupling partner (e.g., an oligonucleotide) can be labeled with a benzylguanine “BG” label then reacted with the SNAP tag to create an irreversible linkage between the two binding partners. In the example described below and depicted in FIG. 5A, a recombinant Protein A-SNAP fusion protein was prepared using standard recombinant methodology and coupled to a “BG” labeled oligonucleotide.

A protein-DNA conjugate can be useful in a variety of applications. In one such application, the manufacture of a protein array by tethering proteins to a glass slides via complementary oligonucleotides has been described. A SNAP tag fusion of a recombinant green fluorescent protein was linked to one oligonucleotide, and the complementary oligonucleotide was UV crosslinked onto the glass slide. The two oligonucleotides were then hybridized to create the protein array. In the same manner two proteins labeled with complementary oligonucleotides could be linked.

With regard to the present invention, directed nucleic acid linkage to proteins for tethering or detection is exemplified in this Example, and depicted in FIG. 5. A single copy of an oligonucleotide can be linked to any recombinant protein of choice (e.g., polymerases, topoisomerases, proteins A, G, and L, restriction enzymes, and ligases) using SNAP-tag technology (Covalys). This oligonucleotide can be used to hybridize or “tether” the recombinant protein to another oligonucleotide of a complementary sequence. This oligonucleotide can also be used directly for detection in QPCR. Restriction enzyme sites can also be introduced for cleavage if desired.

With reference to FIG. 5A, the Figure shows a schematic of the SNAP tag technology. The SNAP tag can be utilized to covalently attach any molecule labeled with the benzylguanine (BG) substrate to any recombinant protein of choice. In this example, an oligonucleotide was labeled with BG then covalently coupled to recombinant Protein A-SNAP (Protein A binds IgG). Use of the technology for creation of a molecule for use in the present invention is depicted in FIG. 5B.

More specifically, FIG. 5B depicts a silver stained acrylamide gel that demonstrates the SNAP tag coupling of an 80mer oligonucleotide (25 kD) to recombinant Protein A-SNAP. The lane on the left marked “−” is unlabeled Protein A-SNAP. The lane on the right marked “+” has been labeled with the oligonucleotide. Arrows indicate the increase in the molecular weight of Protein A-SNAP by 25 kD after the oligonucleotide has been coupled. “M” indicates molecular weight markers.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A detection reagent for detecting a substance of interest, said reagent comprising:
a binding portion comprising a member of a specific binding pair;
a catalytic portion comprising a catalyst; and
a linker portion,
wherein the binding portion and catalytic portions are linked via the linker portion, which comprises an engineered cleavage site for separating the binding portion from the catalytic portion.

2. The reagent of claim 1, wherein the binding portion comprises an antibody or portion of an antibody that is specific for the substance of interest.

3. The reagent of claim 1, wherein the catalytic portion comprises a catalyst that can directly catalyze the production of a detectable product.

4. The reagent of claim 1, wherein the catalytic portion is an enzyme or catalytic portion thereof.

5. The reagent of claim 4, wherein the enzyme is a polymerase.

6. The reagent of claim 1, wherein the linker portion comprises a polyamino acid sequence or a polynucleotide sequence that comprises a covalent cleavage site.

7. The reagent of claim 1, wherein the cleavage site is a restriction endonuclease cleavage site.

8. The reagent of claim 1, wherein the cleavage site is a protease cleavage site.

9. The reagent of claim 1, comprising:
a binding portion comprising an antibody;
a catalytic portion comprising a polymerase; and
a linker portion comprising a biotin-streptavidin linkage and a polyamino acid sequence comprising an engineered protease cleavage site for separating the binding portion from the catalytic portion.

10. The reagent of claim 1, comprising:
a binding portion comprising an antibody;
a catalytic portion comprising a polymerase; and
a linker portion comprising a polynucleic acid sequence comprising an engineered endonuclease cleavage site for separating the binding portion from the catalytic portion.

11. A composition comprising the detection reagent of claim 1.

12. A kit comprising the detection reagent of claim 1.

13. A method for detecting a substance of interest, said method comprising:
causing a detection reagent comprising a binding portion, a linker portion, and a catalytic portion to contact a sample containing or suspected of containing a substance of interest under conditions where the reagent can specifically contact the substance of interest, if present;
removing unbound reagent, non-specifically bound reagent, or both;
causing a bond in the linker portion of the detection reagent to break;
physically separating the catalytic portion of the reagent from the binding portion; and
causing the catalytic portion to catalyze a reaction that results, either directly or ultimately, in production of a detectable signal,
wherein production of a detectable signal is indicative of the presence of the substance of interest in the sample.

14. The method of claim 13, further comprising causing the substance of interest to bind to a solid support.

15. The method of claim 13, wherein the method is a method of immuno-PCR.

16. The method of claim 13, wherein the substance of interest is a drug or a substance produced by a living organism.

17. The method of claim 13, wherein the sample is taken from a human or animal or from the environment.

18. The method of claim 13, which is a diagnostic method for detecting a marker for a disease or disorder.

19. The method of claim 13, which is a method for following the progression of a disease or disorder.

20. The method of claim 19, wherein the disease or disorder is one in a patient undergoing treatment for the disease or disorder.

21. The method of claim 13, wherein the binding portion of the detection reagent comprises an antibody or a portion of an antibody that is specific for the substance of interest.

22. The method of claim 13, wherein the catalytic portion of the detection reagent comprises an enzyme that can directly catalyze the production of a detectable product.
